(19) United States
(12) Patent Application Publication
(10) Pub. No.: US 2023/0181753 A1
(43) Pub. Date: Jun. 15, 2023

(54) NEW METHOD TO TREAT CUTANEOUS T-CELL LYMPHOMAS AND TFH DERIVED LYMPHOMAS

(71) Applicants: INSERM (INSTITUT NATIONAL DE LA SANTE ET DE LA RECHERCHE MEDICALE), Paris (FR); UNIVERSITÉ D'AIX MARSEILLE, Marseille Cedex 07 (FR); CENTRE NATIONAL DE LARECHERCHE SCIENTIFIQUE (CNRS), Paris (FR); INSTITUT JEAN PAOLI & IRENE CALMETTES, Marseille (FR)

(72) Inventors: Daniel OLIVE, Marseille (FR); Florent AMATORE, Marseille Cedex 9 (FR)

(21) Appl. No.: 17/998,584

(22) PCT Filed: May 12, 2021
(86) PCT No.: PCT/EP2021/062650

§ 371 Date: Nov. 11, 2022

(30) Foreign Application Priority Data
May 12, 2020 (EP) 20305480.4
May 28, 2020 (EP) 20305557.9

(51) Int. Cl. A61K 47/68; C07K 16/28; A61K 38/08; A61P 35/00

(52) U.S. Cl. CPC A61K 47/6811; C07K 16/2818; A61K 47/6849; A61K 38/08; A61P 35/00

(57) ABSTRACT

The present invention relates to the treatment of cutaneous T-cell lymphomas (CTCL) and T_{FH} derived lymphomas. In this study, the inventors showed the expression of ICOS by tumor cells in the skin of patients with MF and SS (CTCL) at different stages of the disease, and in the blood of patients with SS. The idea was thus to kill these tumor cells using ADC-antibodies specifics to ICOS. Thanks to cell lines murine xenograft models and Patient Derived Xenografts (PDXs), they showed the efficacy of such anti-ICOS ADCs on T_{FH}-derived lymphomas, such as CTCL and AITL. Thus, the present invention relates to an anti-ICOS antibody for use in the treatment of a cutaneous T-cell lymphomas (CTCL) and/or a TFH derived lymphoma in a subject in need thereof.

### FIELD OF THE INVENTION

The present invention relates to an anti-ICOS antibody for use in the treatment of a cutaneous T-cell lymphomas (CTCL) and/or a TFH derived lymphoma in a subject in need thereof.

### BACKGROUND OF THE INVENTION

Primary cutaneous T-cell lymphomas (CTCLs) account for approximately two-thirds of all primary cutaneous lymphomas, (1) with mycosis fungoides (MF) and Sézary syndrome (SS) the most common subtypes (1). Both MF and SS are characterized by a monoclonal proliferation of mature T-helper lymphocytes in the skin. Tumor cells in MF are classically CD3⁺CD4⁺CD8⁻, with frequent loss of CD7 (2). Sézary cells (circulating malignant lymphocytes) are CD4⁺CD7⁻, and/or CD4⁺CD26⁻, and frequently express CD158k (KIR3DL2) (3). CD158k is the most sensitive marker for detection of Sézary cells in the blood and skin (4-6). Programmed Death-1 (PD-1) is also expressed by the neoplastic T-cells in the skin and blood (7,8) and represents a useful marker for the diagnosis of SS skin lesions (9) However, the phenotype of Sézary cells varies greatly between patients (5,10).

Advanced CTCLs remain an unmet medical need. Brentuximab vedotin (BV) (11) an anti-CD30 antibody-drug conjugate (ADC) linked to monomethyl auristatin E (MMAE), do not deliver significant long-term improvements in patient outcomes. More recently, mogamulizumab (12) and anti-KIR3DL213 provided encouraging results but new targeted therapies are needed.

In lymphomagenesis, tumoral T-cells can overexpress both costimulatory receptors that allow them to survive, proliferate, and resist apoptosis, and coinhibitory receptors that are associated with their functional exhaustion (14,15). In CTCLs, tumor growth may be driven by both costimulatory and coinhibitory receptors (16). On the one hand, tumoral and non-tumoral CD4 T-cells in CTCLs express a wide range of coinhibitory receptors, such as PD-1 (16). On the other hand, in a small cohort of patients with MF, immunohistochemical analysis also revealed the upregulation of costimulatory receptors such as inducible T cell costimulator (ICOS) on the surface of malignant T-cells (17). More recently, analysis of epidermal and dermal explant cultures of skin biopsies from patients with CTCL revealed that there were more ICOS+ T-cells in CTCL samples than in samples from healthy donor skin without, however, specifying the tumoral or reactive nature of these lymphocytes (16).

ICOS (CD278, AILTIM, H4) is a costimulatory receptor for T-cell enhancement and a member of the B7/CD28 receptor superfamily (18). It is upregulated on activated T lymphocytes (CD4 and CD8 effector, T follicular helper [TFH], regulatory T cells [Tregs]). Naïve T-cells express low levels of ICOS but its expression is rapidly induced after T-cell receptor engagement. Its unique ligand, ICOSL, is expressed by antigen-presenting cells, B-cells, and many non-hematopoietic cells (19). The engagement of ICOS by its ligand induces proliferation, survival, differentiation, and cytokine production in order to potentiate the antigen-specific immune response.

The high level of ICOS expression by T_{F}H-derived tumor cells has been known for around 20 years (20,21). Malignant cells in angioimmunoblastic T-cell lymphoma (AITL) and primary cutaneous CD4⁺ small/medium T-cell lympho-proliferative disorder (PCSMTLPD) widely express ICOS. Moreover, activated Tregs also express ICOS (19). and ICOS⁺ Tregs exhibit a higher immunosuppressive capacity than ICOS⁻ Tregs (22). Recently, Geskin et al (23) identified a high level of Tregs in the blood of patients with SS. The inhibitory impact of mogamulizumab on Tregs partly explains its efficacy in SS (24).

ICOS is therefore a promising therapeutic target due to its wide expression in several peripheral T-cell lymphoma (PTCL), likely by both malignant T-cells and Tregs.

### SUMMARY OF THE INVENTION

In this study, the inventors showed the expression of ICOS by tumor cells in the skin of patients with MF and SS (CTCL) at different stages of the disease, and in the blood of patients with SS. The idea was thus to kill these tumor cells using ADC-antibodies specifics to ICOS. Thanks to cell lines murine xenograft models and Patient Derived Xenografts (PDXs), they showed the efficacy of such anti-ICOS ADCs on T_{FH}-derived lymphomas, such as CTCL and AITL.

Thus, the present invention relates to an anti-ICOS antibody for use in the treatment of a cutaneous T-cell lymphomas (CTCL) and/or a TFH derived lymphoma in a subject in need thereof. In particular, the present invention is defined by the claims.

### DETAILED DESCRIPTION OF THE INVENTION

The present invention relates to an anti-ICOS antibody for use in the treatment of a cutaneous T-cell lymphomas (CTCL) and/or a TFH derived lymphoma in a subject in need thereof.

As used herein, the terms “anti-ICOS antibody” denotes a monoclonal antibody which can target ICOS or ICOS-ligand (the ICOS pathway). Such antibody can bind to ICOS or ICOS-L and block the activity of the ICOS pathway for example the activation of the he PI3K/AKT signaling pathway and the enhancement of the anti-tumor T cell responses of said pathway or just bind to ICOS, ICOS-L or the recombinant protein ICOS-L.

According to the invention, the ICOS-L can be the Recombinant human B7-H2 Fc Chimera Protein, CF.

As used herein, the terms “ICOS” for “Inductible T cell costimulator” (CD278, AILIM, H4) refer to a transmembrane homodimeric glycoprotein of 55 to 60 kDa which presents an IgV type domain in its extracellular part and a tyrosine within an YMFM motif in its cytoplasmic part. It has been shown that ICOS engagement with its unique ligand (ICOSL, CD275, B7-H2, B7h, B7RP-1) induces the phosphorylation of the tyrosine in the cytoplasmic part of ICOS. Said phosphorylation is responsible for the recruitment of the p85 PI3K regulatory subunit, which activates the PI3K/AKT signaling pathway. ICOS engagement is also described to induce the expression of CD40L at the cell surface. CD40L is known to have an important effect in the cooperation between T lymphocytes and B lymphocytes. ICOS, as a member of 20 the costimulatory B7-1/B7-2-CD28/CTLA-4 family, is rapidly induced after TCR engagement on conventional T cells (Tconv CD4⁺, CD8⁺ subsets) as well as on Treg. ICOS shows a dualistic behaviour in oncogenesis, as it can both enhance anti-tumor T cell responses and support tumor development through Tregs, as in patients suffering from melanoma or breast cancer. Its Entrez Gene ID number is 29851.

As used herein, the term “T_{FH} derived lymphoma” has its general meaning in the art and denotes an aggressive mature peripheral T-cell lymphoma originating from the T_{FH} cells, presenting with generalized lymphadenopathy and hepatosplenomegaly. It is characterized by a polymorphous lymph node infiltrate showing a marked increase in follicular dendritic cells (FDCs) and high endothelial venules (HEVs) and systemic involvement. T_{FH} derived lymphoma includes the Angioimmunoblastic T-cell Lymphoma (AITL), the primary cutaneous CD4+ small/medium T-cell lymphoma (PCSMLPD) and neoplasms (see for example Shimin Hu M D et al. 2012).

As used herein, the term “Cutaneous T-cell Lymphomas (CTCL))” has its general meaning in the art and denotes a class of non-Hodgkin lymphoma, which is a type of cancer of the immune system. Unlike most non-Hodgkin lymphomas (which are generally B cell related), CTCL is caused by a mutation of T cells. The tumor T cells in the body initially migrate to the skin, causing various lesions to appear. These lesions change shape as the disease progresses, typically beginning as what appears to be a rash which can be very itchy and eventually forming plaques and tumors before spreading to other parts of the body.

According to the invention, the CTCL can be a primary cutaneous T-cell lymphomas and regroup the following diseases: Mycosis Fungoides (MF) and MF variants (folliculotropic, pagetoid reticulosis, granulomatous slack skin), Sézary syndrome (SS) Adult T-cell leukemia/lymphoma, Primary cutaneous CD30⁺ lymphoproliferative diseases (cutaneous anaplastic T-cell lymphoma and lymphomatoid papulosis), Subcutaneous panniculitis-like T-cell lymphoma, Extranodal NK/T-cell lymphoma (nasal type), Primary cutaneous g/d T-cell lymphoma, CD8⁺ AECTCL, Primary cutaneous CD4⁺ small/medium T-cell lymphoproliferative disorder, Primary cutaneous acral CD8⁺ T-cell lymphoma, Primary cutaneous peripheral T-cell lymphoma NOS.

Particularly, the CTCL is a mycosis fungoides or a Sézary syndrome.

As used herein, the term “subject” denotes a mammal, such as a rodent, a feline, a canine, and a primate. Particularly, the subject according to the invention is a human. More particularly, the subject according to the invention suffers from a cutaneous T-cell lymphomas (CTCL) or a T_{FH} derived lymphoma.

### Antibodies of the Invention

The inventors showed that different anti-ICOS antibodies used in ADC or ADCC/ADCP could be useful to treat a cutaneous T-cell lymphomas (CTCL) and/or a T_{FH} derived lymphoma in a subject in need thereof.

Thus, an anti-ICOS antibody could be any antibody which target ICOS or ICOS-L.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

As used herein, the term “specificity” refers to the ability of an antibody to detectably bind an epitope presented on an antigen, such as ICOS, while having relatively little detectable reactivity with non-ICOS proteins or structures. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is ICOS).

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope.

The antibodies of the present invention are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

According to the invention, the terms used in plural or in singular are used in an equivalent manner.

Particularly, the anti-ICOS of the invention can be an antibody as described in the patent application WO2008137915 or WOO187981.

Particularly, the anti-ICOS of the invention can be one of the antibodies GSK3359609, JTX-2011, MEDI-570 or KY1044 as described in Solinas et al. 2019.

Particularly, the anti-ICOS antibody of the invention can be one of the antibodies described in the patent application WO2012131004 (53.3 mab, 88.2 mab, 92.17 mab, 145.1 mab and 314.8 mab and the derivatives thereof).

As used herein, the expression “derivative of an antibody” refers to an antibody which comprises the 6 CDRs of said antibody.

As used herein, the expression “53.3 mAb” or “Icos 53-3” refers to a monoclonal antibody directed against ICOS deposited at the CNCM on Jul. 2, 2009 under the accession number CNCM I-4176. Said antibody is an agonist of ICOS. The expression “a derivative of 53.3 mAb” refers to an anti-ICOS antibody which comprises the 6 CDRs of 53.3 mAb.

As used herein, the expression “88.2 mAb” or “Icos 88-2” refers to a monoclonal antibody directed against ICOS deposited at the CNCM on Jul. 2, 2009 under the accession number CNCM I-4177. Said antibody is an agonist of ICOS. The expression “a derivative of 88.2 mAb” refers to an anti-ICOS antibody which comprises the 6 CDRs of 88.2 mAb.

As used herein, the expression “92.17 mAb” or “Icos 92-17” refers to a monoclonal antibody directed against ICOS deposited at the CNCM on Jul. 2, 2009 under the accession number CNCM I-4178. Said antibody is an agonist of ICOS. The expression “a derivative of 92.17 mAb” refers to an anti-ICOS antibody which comprises the 6 CDRs of 92.17 mAb.

As used herein, the expression “145.1 mAb” or “Icos 145-1” refers to a monoclonal antibody directed against ICOS deposited at the CNCM on Jul. 2, 2009 under the accession number CNCM I-4179. Said antibody is an antagonist of ICOS. The expression “a derivative of 145.1 mAb” refers to an anti-ICOS antibody which comprises the 6 CDRs of 145-1 mAb.

As used herein, the expression “314.8 mAb” or “Icos 314-8” refer to a monoclonal antibody directed against ICOS deposited to CNCM on Jul. 2, 2009 under the accession number CNCM I-4180. The expression “a derivative of 314.8 mAb” refers to an anti-ICOS antibody which comprises the 6 CDRs of 314.8 mAb.

Particularly, the anti-ICOS antibody of the invention can be the 88.2 antibody with the followings CDRs (table 1):

TABLE 1
CDRs of the 88.2 antibody

| | Amino acids | Amino acids |
|---|---|---|
| | sequences | sequences |
| | (IMGT | (Kabat |
| | nomenclature) | nomenclature) |
| H-CDR1 | GYSFTSYW | SYWIN |
| | (SEQ ID NO: 1) | (SEQ ID NO: 21) |
| H-CDR2 | IYPSDSYT | NIYPSDSYTNY |
| | (SEQ ID NO: 2) | NQMFKD |
| | | (SEQ ID NO : 22) |
| H-CDR3 | TRWNLSYYFDNNYYLD | WNLSYYFDNNYYLDY |
| | Y (SEQ ID NO: 3) | (SEQ ID NO : 23) |
| L-CDR1 | KSLLHSNG | RSSKSLLHS |
| | NTY | NGNTYLY |
| | (SEQ ID NO: 4) | (SEQ ID NO: 24) |
| L-CDR2 | RMS | RMSNLAS |
| | (SEQ ID NO: 5) | (SEQ ID NO: 25) |
| L-CDR3 | MQHLEYPWT | MQHLEYPWT |
| | (SEQ ID | (SEQ ID |
| | NO: 6) | NO: 26) |

Amino acids sequence of the heavy chain
(H) of the 88.2 mAh (SEQ ID NO: 7):
QVQLQQPGAELVRPGASVKLSCKASGYSFTSYWIN
WVKQRPGQGLEWIGNIYPSDSYTNYNQMFKDKATL
TVDKSSNTAYMQLTSPTSEDSAVYYCTRWNLSYYF
DNNYYLDYWGQGTTLTVSS
Amino acids sequence of the light chain
(L) of the 88.2 mAh (SEQ ID NO: 8):
DIVMTQAAPSVPVTPGESVSISCRSSKSLLHSNGN
TYLYWFLQRPGQSPQLLIYRMSNLASGVPDRFSGS
GSGTAFTLRISRVEAEDVGVYYCMQHLEYPWTFGG
GTKLEI

Particularly, the anti-ICOS antibody of the invention can be the 314.8 antibody with the followings CDRs (table 2):

TABLE 2
CDRs of the 314.8 antibody

| | Amino acids | Amino acids |
|---|---|---|
| | sequences | sequences |
| | (IMGT | (Kabat |
| | nomenclature) | nomenclature) |
| H-CDR1 | GYTFTTYW | TYWMH |
| | (SEQ ID | (SEQ ID |
| | NO: 9) | NO: 27) |
| H-CDR2 | IDPSDSYV | EIDPSDSY |
| | (SEQ ID NO: | VNYNQNFK |
| | 10) | G (SEQ ID |
| | | NO: 28) |
| H-CDR3 | ARSPDYYG | SPDYYGTS |
| | TSLAWFDY | LAWFDY |
| | (SEQ ID | (SEQ ID |
| | NO: 11) | NO: 29) |
| L-CDR1 | KSPLHSNGNIY | RSSKSPLH |
| | (SEQ ID | SNGNIYLY |
| | NO: 12) | (SEQ ID |
| | | NO: 30) |
| L-CDR2 | RMS | RMSNLAS |
| | (SEQ ID | (SEQ ID |
| | NO: 13) | NO: 31) |
| L-CDR3 | MQHLEYPYT | MQHLEYPYT |
| | (SEQ ID | (SEQ ID |
| | NO: 14) | NO: 32) |

Amino acids sequence of the heavy chain (H)
of the 314.8 mAh (SEQ ID NO: 15):
QVQLQQPGTELMKPGASVKLSCKASGYTFTTYWM
HWVKQRPGQGLEWIGEIDPSDSYVNYNQNFKGKA
TLTVDKSSSTAYIQLSSLTSEDSAVYFCARSPDY
YGTSLAWFDYWGQGTLVTVST
Amino acids sequence of the light chain (L)
of the 314.8 mAh (SEQ ID NO: 16):
DIVMTQAAPSVPVTPGESVSISCRSSKSPLHSNG
NIYLYWFLQRPGQSPQLLIYRMSNLASGVPDRFS
GSGSGTTFTLKISRVEAEDVGVYYCMQHLEYPYT
FGGGTKLEIK

The amino acid residues of the antibody of the invention could be numbered according to the IMGT or KABAT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999).; Lefranc, M.-P., Pommie, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003).). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. (http://www.imgt.org/IMGTScientificChart/Nomenclature/IMGT-FRCDRdefinition.html)

The residues in antibody variable domains can be conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. (http://www.bioinf.org.uk/abs/#cdrdef)

The present invention thus provides antibodies comprising functional variants of the VL region, VH region, or one or more CDRs of the antibodies of the invention. A functional variant of a VL, VH, or CDR used in the context of a monoclonal antibody of the present invention still allows the antibody to retain at least a substantial proportion (at least about 50%, 60%, 70%, 80%, 90%, 95% or more) of the affinity/avidity and/or the specificity/selectivity of the parent antibody and in some cases such a monoclonal antibody of the present invention may be associated with greater affinity, selectivity and/or specificity than the parent Ab. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent Ab. The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:

Aliphatic residues I, L, V, and M

Cycloalkenyl-associated residues F, H, W, and Y

Hydrophobic residues A, C, F, G, H, I, L, M, R, T, V, W, and Y

Negatively charged residues D and E

Polar residues C, D, E, H, K, N, Q, R, S, and T

Positively charged residues H, K, and R

Small residues A, C, D, G, N, P, S, T, and V

Very small residues A, G, and S

Residues involved in turn A, C, D, E, G, H, K, N, Q, R, S, P, and formation T

Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the antibodies of the invention. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 90% of identity with a second amino acid sequence means that the first sequence has 90; 91; 92; 93; 94; 95; 96; 97; 98; 99; or 100% of identity with the second amino acid sequence.

In some embodiments, the antibody of the present invention is an antibody having a heavy chain comprising i) the H-CDR1 of the 53.3 mab, 88.2 mab, 92.17 mab, 145.1 mab or 314.8 mab, ii) the H-CDR2 of 53.3 mab, 88.2 mab, 92.17 mab, 145.1 mab or 314.8 mab and iii) the H-CDR3 of 53.3 mab, 88.2 mab, 92.17 mab, 145.1 mab or 314.8 mab and a light chain comprising i) the L-CDR1 of 53.3 mab, 88.2 mab, 92.17 mab, 145.1 mab or 314.8 mab, ii) the L-CDR2 of 53.3 mab, 88.2 mab, 92.17 mab, 145.1 mab or 314.8 mab and iii) the L-CDR3 of 53.3 mab, 88.2 mab, 92.17 mab, 145.1 mab or 314.8 mab.

In some embodiments, the antibody of the present invention is an antibody having a heavy chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:7 or 15 and a light chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:8 or 16.

In some embodiments, the antibody of the present invention is an antibody having a heavy chain identical to SEQ ID NO:7 or 15 and a light chain identical to SEQ ID NO:8 or 16.

In one embodiment, the monoclonal antibody of the invention is a chimeric antibody, particularly a chimeric mouse/human antibody.

Thus, the present invention relates to an anti-ICOS chimeric antibody for use in the treatment of a cutaneous T-cell lymphomas (CTCL) and/or a T_{FH} derived lymphoma in a subject in need thereof.

According to the invention, the term “chimeric antibody” refers to an antibody which comprises a VH domain and a VL domain of a non-human antibody, and a CH domain and a CL domain of a human antibody.

In some embodiments, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204,244).

According to the invention the anti-ICOS antibody can be a chimeric antibody of the antibodies described above and particularly the antibodies 53.3 mab, 88.2 mab, 92.17 mab, 145.1 mab and 314.8 mab.

In another embodiment, the monoclonal antibody of the invention is a humanized antibody. In particular, in said humanized antibody, the variable domain comprises human acceptor frameworks regions, and optionally human constant domain where present, and non-human donor CDRs, such as mouse CDRs.

Thus, the present invention relates to an anti-ICOS humanized antibody for use in the treatment of a cutaneous T-cell lymphomas (CTCL) and/or a T_{FH} derived lymphoma in a subject in need thereof.

In one embodiment, the humanized antibody can be derived from a chimeric antibody (obtained from the antibody of the invention).

In another embodiment, the monoclonal antibody of the invention is a caninized or primatized based on the same methods of humanization.

According to the invention, the term “humanized antibody” refers to an antibody having variable region framework and constant regions from a human antibody but retains the CDRs of a previous non-human antibody.

The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

According to the invention the anti-ICOS antibody can be a humanized antibody of the antibodies described above and particularly the antibodies 53.3 mab, 88.2 mab, 92.17 mab, 145.1 mab and 314.8 mab.

In some embodiments the antibody of the invention is a human antibody.

Thus, the present invention relates to an anti-ICOS human antibody for use in the treatment of a cutaneous T-cell lymphomas (CTCL) and/or a T_{FH} derived lymphoma in a subject in need thereof.

As used herein the term “human antibody is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Human antibodies can be produced using various techniques known in the art. Human antibodies are described generally in van Dijk and van de Winkel, cur. Opin. Pharmacol. 5; 368-74 (2001) and lonberg, cur. Opin.Immunol. 20; 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23; 1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application publication No. US 2007/0061900, describing VELOCIMOUSE® technology. Human variable regions from intact antibodies generated by such animals may be further modified, e.g., by combining with a different human constant region Human antibodies can also be made by hybridoma-based methods. Human myeloma and mouse-human heteromyeloma cell lines for the production of human monoclonal antibodies have been described. (See, e.g., Kozbor J. Immunol., 13: 3001 (1984); Brodeur et al., Monoclonal Antibody Production Techniques and Applications, pp. 51-63 (Marcel Dekker, Inc., New York, 1987); and Boerner et al., J. Immunol., 147: 86(1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human igM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005). Fully human antibodies can also be derived from phage-display libraries (as disclosed in Hoogenboom et al., 1991, J. Mol. Biol. 227:381; and Marks et al., 1991, J. Mol. Biol. 222:581). Phage display techniques mimic immune selection through the display of antibody repertoires on the surface of filamentous bacteriophage, and subsequent selection of phage by their binding to an antigen of choice. One such technique is described in PCT publication No. WO 99/10494. Human antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In one embodiment, the antibody of the invention is an antigen biding fragment (here ICOS biding fragment) selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL or VH chains as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:7 or 15 and/or SEQ ID NO:8 or 16.

Thus, the present invention relates to an ICOS biding fragment for use in the treatment of a T_{FH} derived lymphoma in a subject in need thereof.

The term “antigen binding fragment” of an antibody, as used herein, refers to one or more fragments of an intact antibody that retain the ability to specifically binds to a given antigen (e.g., [antigen]). Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

The Fab of the present invention can be obtained by treating an antibody which specifically reacts with [antigen] with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with [antigen] with a protease, pepsin. Also, the F(ab′)2 can be produced by binding Fab′ described below via a thioether bond or a disulfide bond.

The Fab′ of the present invention can be obtained treating F(ab′)2 which specifically reacts with [antigen] with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

According to the invention the anti-ICOS antibody can be a ICOS biding fragment of the antibodies described above and particularly the antibodies 53.3 mab, 88.2 mab, 92.17 mab, 145.1 mab and 314.8 mab.

### Single Domain Antibody

In a particular embodiment, the anti-ICOS antibody is an anti-ICOS single domain antibody.

Thus, the present invention relates to an anti-ICOS single domain antibody for use in the treatment of a cutaneous T-cell lymphomas (CTCL) and/or a T_{FH} derived lymphoma in a subject in need thereof.

As used herein the term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus yet another consequence of small size is that a nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

Camel Ig can be modified by genetic engineering to yield a small protein having high affinity for a target, resulting in a low molecular weight antibody-derived protein known as a “nanobody” or “VHH”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. In certain embodiments herein, the camelid antibody or nanobody is naturally produced in the camelid animal, i.e., is produced by the camelid following immunization with [antigen] or a peptide fragment thereof, using techniques described herein for other antibodies. Alternatively, the [antigen]-binding camelid nanobody is engineered, i.e., produced by selection for example from a library of phage displaying appropriately mutagenized camelid nanobody proteins using panning procedures with [antigen] as a target.

In some embodiments, the single domain antibody is a “humanized” single domain antibody.

As used herein the term “humanized” refers to a single domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favourable properties of single domain antibodies of the invention. The one skilled in the art is able to determine and select suitable humanizing substitutions or suitable combinations of humanizing substitutions.

A further aspect of the invention refers to a polypeptide comprising at least one single domain antibody of the invention.

Typically, the polypeptide of the invention comprises a single domain antibody of the invention, which is fused at its N terminal end, at its C terminal end, or both at its N terminal end and at its C terminal end to at least one further amino acid sequence, i.e. so as to provide a fusion protein. According to the invention the polypeptides that comprise a sole single domain antibody are referred to herein as “monovalent” polypeptides. Polypeptides that comprise or essentially consist of two or more single domain antibodies according to the invention are referred to herein as “multivalent” polypeptides.

According to the invention, the single domain antibodies and polypeptides of the invention may be produced by conventional automated peptide synthesis methods or by recombinant expression. General principles for designing and making proteins are well known to those of skill in the art. The single domain antibodies and polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The single domain antibodies and polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

### Acid Nucleic, Vector and Host Cell

A further object of the invention relates to a nucleic acid molecule encoding an antibody according to the invention. More particularly the nucleic acid molecule encodes a heavy chain or a light chain of an antibody of the present invention.

Thus, the present invention relates a nucleic acid molecule encoding an antibody according to the invention for use in the treatment of a cutaneous T-cell lymphomas (CTCL) and/or a T_{FH} derived lymphoma in a subject in need thereof.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSGl beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

Nucleic acids sequence of the heavy chain (H)
of the 88.2 mAh is (SEQ ID NO: 17):
CAGGTCCAACTGCAGCAGCCTGGGGCTGAGCTGGT
GAGGCCTGGGGCTTCAGTGAAGCTGTCCTGCAAGG
CTTCTGGCTACAGTTTCACCAGCTACTGGATAAAC
TGGGTGAAGCAGAGGCCTGGACAAGGCCTTGAGTG
GATCGGAAATATTTATCCTTCTGATAGTTATACTA
ACTACAATCAAATGTTCAAGGACAAGGCCACATTG
ACTGTAGACAAATCCTCCAACACAGCCTACATGCA
GCTCACCAGCCCGACATCTGAGGACTCTGCGGTCT
ATTACTGTACAAGATGGAATCTTTCTTATTACTTC
GATAATAACTACTACTTGGACTACTGGGGCCAAGG
CACCACTCTCACAGTCTCCTCA
Nucleic acids sequence of the light chain (L)
of the 88.2 mAh is (SEQ ID NO: 18):
GATATTGTGATGACTCAGGCTGCACCCTCTGTACC
TGTCACTCCTGGAGAGTCAGTATCCATCTCCTGCA
GGTCTAGTAAGAGTCTCCTGCATAGTAATGGCAAC
ACTTACTTGTATTGGTTCCTGCAGAGGCCAGGCCA
GTCTCCTCAACTCCTGATATATCGGATGTCCAACC
TTGCCTCAGGAGTCCCAGACAGGTTCAGTGGCAGT
GGGTCAGGAACTGCTTTCACACTGAGAATCAGTAG
AGTGGAGGCTGAGGATGTGGGTGTTTATTACTGTA
TGCAACATCTAGAATATCCGTGGACGTTCGGTGGA
GGCACCAAGCTGGAAATCAAA
Nucleic acids sequence of the heavy chain (H)
of the 314.8 mAh is (SEQ ID NO: 19):
CAGGTCCAACTACAGCAGCCTGGGACTGAACTTAT
GAAGCCTGGGGCTTCAGTGAAGCTGTCCTGCAAGG
CTTCTGGCTACACCTTCACCACCTACTGGATGCAC
TGGGTGAAGCAGAGGCCTGGACAAGGCCTTGAGTG
GATCGGAGAGATTGATCCTTCTGATAGTTATGTTA
ACTACAATCAAAACTTTAAGGGCAAGGCCACATTG
ACTGTAGACAAATCCTCCAGCACAGCCTACATACA
GCTCAGCAGCCTGACATCTGAGGACTCTGCGGTCT
ATTTTTGTGCGAGATCCCCTGATTACTACGGTACT
AGTCTTGCCTGGTTTGATTACTGGGGCCAAGGGAC
TCTGGTCACTGTCTCTACA
Nucleic acids sequence of the light chain (L)
of the 314.8 mAh is (SEQ ID NO: 20):
GATATTGTGATGACTCAGGCTGCACCCTCTGTACC
TGTCACTCCTGGAGAGTCAGTATCCATCTCCTGCA
GGTCTAGTAAGAGTCCCCTGCATAGTAACGGCAAC
ATTTACTTATATTGGTTCCTGCAGAGGCCAGGCCA
GTCTCCTCAGCTCCTGATATATCGGATGTCCAACC
TTGCCTCAGGAGTCCCAGACAGGTTCAGTGGCAGT
GGGTCAGGAACTACTTTCACACTGAAAATCAGTAG
AGTGGAGGCTGAGGATGTGGGTGTTTATTACTGTA
TGCAACATCTAGAATATCCGTACACGTTCGGAGGG
GGGACCAAGCTGGAAATAAAA

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### Antibodies which Compete with the Antibodies of the Invention

In another aspect, the invention provides an antibody that competes for binding to ICOS with an antibody of the invention.

Thus, the present invention relates an antibody that competes for binding to ICOS with an antibody of the invention for use in the treatment of a cutaneous T-cell lymphomas (CTCL) and/or a T_{FH} derived lymphoma in a subject in need thereof.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, N.J.) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard ICOS binding assays. The ability of a test antibody to inhibit the binding of antibodies of the present invention to ICOS demonstrates that the test antibody can compete with that antibody for binding to ICOS; such an antibody may, according to non-limiting theory, bind to the same or a related (e.g., a structurally similar or spatially proximal) epitope on ICOS as the antibody with which it competes. Thus, another aspect of the invention provides antibodies that bind to the same antigen as, and compete with, the antibodies disclosed herein. As used herein, an antibody “competes” for binding when the competing antibody inhibits ICOS binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of ICOS. In some embodiments, the epitopes to which the present antibodies or antigen binding fragments bind are linear epitopes. In other embodiments, the epitopes to which the present antibodies or antigen binding fragments bind are non-linear, conformational epitopes.

The antibodies of the invention may be assayed for specific binding by any method known in the art. Many different competitive binding assay format(s) can be used for epitope binding. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York).

### Antibody Engineering

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell—epitopes to thereby reduce the potential immunogenicity of the antibody. This approach is also referred to as “deimmunization” and is described in further detail in U.S. Patent Publication No. 20030153043 by Carr et al.

In some embodiments, the glycosylation of the antibody is modified. Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

In some embodiments, some mutations are made to the amino acids localized in aggregation “hotspots” within and near the first CDR (CDR1) to decrease the antibodies susceptibility to aggregation (see Joseph M. Perchiacca et al., Proteins 2011; 79:2637-2647).

The antibody of the present invention may be of any isotype. The choice of isotype typically will be guided by the desired effector functions. IgG1 and IgG3 are isotypes that mediate such effectors functions as ADCC or CDC, when IgG2 and IgG4 don't or in a lower manner. Either of the human light chain constant regions, kappa or lambda, may be used. If desired, the class of a monoclonal antibody of the present invention may be switched by known methods. Typical, class switching techniques may be used to convert one IgG subclass to another, for instance from IgG1 to IgG2. Thus, the effector function of the monoclonal antibodies of the present invention may be changed by isotype switching to, e.g., an IgG1, IgG2, IgG3, IgG4, IgD, IgA, IgE, or IgM antibody for various therapeutic uses.

In some embodiments, the antibody of the present invention is a full-length antibody. In some embodiments, the full-length antibody is an IgG1 antibody. In some embodiments, the full-length antibody is an IgG3 antibody.

Thus, the invention also relates to an anti-ICOS IgG1 antibody for use in the treatment of a cutaneous T-cell lymphomas (CTCL) and/or a T_{FH} derived lymphoma in a subject in need thereof.

In some embodiments, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some embodiments, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by ldusogie et al.

In some embodiments, one or more amino acid residues are altered to thereby alter the ability of the antibody to fix complement. This approach is described further in PCT Publication WO 94/29351 by Bodmer et al.

In some embodiments, the Fc region is modified to increase the ability of the antibody to mediate antibody dependent cellular cytotoxicity (ADCC) or Antibody-dependent cellular phagocytosis (ADCP) and/or to increase the affinity of the antibody for an Fc receptor by modifying one or more amino acids. This approach is described further in PCT Publication WO 00/42072 by Presta. Moreover, the binding sites on human IgGI for FcγRI, FcγRII, FcγRIII and FcRn have been mapped and variants with improved binding have been described (see Shields, R. L. et al., 2001 J. Biol. Chen. 276:6591-6604, WO2010106180).

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

As used herein, the term “effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the monoclonal antibodies of the present invention can be produced in yeasts or filamentous fungi engineered for mammalian-like glycosylation pattern and capable of producing antibodies lacking fucose as glycosylation pattern (see for example EP1297172B1).

In another embodiment, the antibody is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Antibodies with increased half-lives and improved binding to the neonatal Fc receptor (FcRn), which is responsible for the transfer of maternal IgGs to the foetus (Guyer et al., J. Immunol. 117:587 (1976) and Kim et al., J. immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311,312, 317, 340, 356, 360, 362, 376, 378, 380, 382, 413, 424, or 434, e.g., substitutions of Fc region residue 434 (U.S. Pat. No. 7,371,826).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

In certain embodiments of the invention antibodies have been engineered to remove sites of deamidation. Deamidation is known to cause structural and functional changes in a peptide or protein. Deamidation can result in decreased bioactivity, as well as alterations in pharmacokinetics and antigenicity of the protein pharmaceutical. (Anal Chem. 2005 Mar. 1; 77(5):1432-9).

In a particular embodiment, the invention relates to an anti-ICOS antibody for use in the treatment of a cutaneous T-cell lymphomas (CTCL) and/or a T_{FH} derived lymphoma in a subject in need thereof wherein said antibody is able to kill the CTCL cells or A T_{FH} derived lymphoma ITL cells by ADCC and/or ADCP.

In others words, the invention relates to an anti-ICOS antibody for use in the treatment of a cutaneous T-cell lymphomas (CTCL) and/or a T_{FH} derived lymphoma in a subject in need thereof wherein said antibody mediates an ADCC activity and/or ADCP activity.

In certain embodiments of the invention the antibodies have been engineered to increase pI and improve their drug-like properties. The pI of a protein is a key determinant of the overall biophysical properties of a molecule. Antibodies that have low pIs have been known to be less soluble, less stable, and prone to aggregation. Further, the purification of antibodies with low pI is challenging and can be problematic especially during scale-up for clinical use. Increasing the pI of the anti-ICOS antibodies of the invention or fragments thereof improved their solubility, enabling the antibodies to be formulated at higher concentrations (>100 mg/ml). Formulation of the antibodies at high concentrations (e.g. >100 mg/ml) offers the advantage of being able to administer higher doses of the antibodies into eyes of patients via intravitreal injections, which in turn may enable reduced dosing frequency, a significant advantage for treatment of chronic diseases including cardiovascular disorders. Higher pIs may also increase the FcRn-mediated recycling of the IgG version of the antibody thus enabling the drug to persist in the body for a longer duration, requiring fewer injections. Finally, the overall stability of the antibodies is significantly improved due to the higher pi resulting in longer shelf-life and bioactivity in vivo. Preferably, the pI is greater than or equal to 8.2.

Glycosylation modifications can also induce enhanced anti-inflammatory properties of the antibodies by addition of sialylated glycans. The addition of terminal sialic acid to the Fc glycan reduces FcγR binding and converts IgG antibodies to anti-inflammatory mediators through the acquisition of novel binding activities (see Robert M. Anthony et al., J Clin Immunol (2010) 30 (Suppl 1):S9-S14; Kai-Ting C et al., Antibodies 2013, 2, 392-414).

### Antiboby Mimetics

In some embodiments, the heavy and light chains, variable regions domains and CDRs that are disclosed can be used to prepare polypeptides that contain antigen binding region that can specifically bind to ICOS. For example, one or more of the CDRs listed in table 1 or 2 can be incorporated into a molecule (e.g., a polypeptide) covalently or noncovalently to make an immunoadhesion. An immunoadhesion may incorporate the CDR(s) as part of a larger polypeptide chain, may covalently link the CDR(s) to another polypeptide chain, or may incorporate the CDR(s) noncovalently. The CDR(s) enable the immunoadhesion to bind specifically to a particular antigen of interest (e.g., ICOS or epitope thereof).

The terms “polypeptide” and “protein” are used interchangeably herein to refer to a polymer of amino acid residues. The terms apply to amino acid polymers in which one or more amino acid residues is an artificial chemical mimetic of a corresponding naturally occurring amino acid, as well to naturally occurring amino acids polymers and non-naturally occurring amino acid polymers. Unless otherwise indicated, a particular polypeptide sequence also implicitly encompasses conservatively modified variants thereof.

In some embodiments, the antigen biding fragment of the invention is grafted into non-immunoglobulin based antibodies also called antibody mimetics selected from the group consisting of an affibody, an affilin, an affitin, an adnectin, an atrimer, an evasin, a DARPin, an anticalin, an avimer, a fynomer, and a versabody.

The term “antibody mimetic” is intended to refer to molecules capables of mimicking an antibody's ability to bind an antigen, but which are not limited to native antibody structures. Examples of such antibody mimetics include, but are not limited to, Adnectins, Affibodies, DARPins, Anticalins, Avimers, and versabodies, all of which employ binding structures that, while they mimic traditional antibody binding, are generated from and function via distinct mechanisms. Antigen biding fragments of antibodies can be grafted into scaffolds based on polypeptides such as Fibronectin type III (Fn3) (see U.S. Pat. No. 6,703,199, which describes fibronectin polypeptide monobodies). An affibody is well known in the art and refers to affinity proteins based on a 58 amino acid residue protein domain, derived from one of the IgG binding domain of staphylococcal protein A. DARPins (Designed Ankyrin Repeat Proteins) are well known in the art and refer to an antibody mimetic DRP (designed repeat protein) technology developed to exploit the binding abilities of non-antibody proteins. Anticalins are well known in the art and refer to another antibody mimetic technology, wherein the binding specificity is derived from lipocalins. Anticalins may also be formatted as dual targeting protein, called Duocalins. Avimers are well known in the art and refer to another antibody mimetic technology, Avimers are derived from natural A-domain containing protein. Versabodies are well known in the art and refer to another antibody mimetic technology, they are small proteins of 3-5 kDa with >15% cysteines, which form a high disulfide density scaffold, replacing the hydrophobic core the typical proteins have. Such antibody mimetic can be comprised in a scaffold. The term “scaffold” refers to a polypeptide platform for the engineering of new products with tailored functions and characteristics.

In one aspect, the invention pertains to generating non-immunoglobulin based antibodies also called antibody mimetics using non-immunoglobulins scaffolds onto which CDRs of the invention can be grafted. Known or future non-immunoglobulin frameworks and scaffolds may be employed, as long as they comprise a binding region specific for the target [antigen] protein.

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprise the entire antigen recognition unit in camel and llama IgG. Because of this structure, the non-immunoglobulin antibody mimics antigen binding properties that are similar in nature and affinity to those of antibodies. These scaffolds can be used in a loop randomisation and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The Ankyrin technology is based on using proteins with Ankyrin derived repeat modules as scaffolds for bearing variable regions which can be used for binding to different targets. The Ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on “A-domains” monomers (2-10) linked via amino acids linkers. Avimers can be created that can bind to the target antigen using the methodology described in, for example, U.S. patent Application publication Nos. 20040175756; 20050053973; 20050048512; and 20060008844.

Affibody affinity ligands are small, simple proteins composed of a three-helix bundle based on the scaffold of one of the IgG-binding domains of protein A. protein A is a surface protein form the bacterium Staphylococcus aureus. This scaffold domain consist of 58 amino acids, 13 of which are randomized to generate affibody librairies with a large number of ligand variants (See e.g., U.S. Pat. No. 5,831,012). Affibody molecules mimic antibodies, they have a molecular weight of 6 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acids residues, being just marginally bigger than a single immunoglobulin domain. The set of four loops, which makes up the binding pocket, shows pronounced structural plasticity and tolerates a variety of side chains. The binding site can can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of “ubiquitin-like” proteins are described in WO2004106368.

Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

The above descriptions of antibody fragment and mimetic technologies is not intended to be comprehensive. A variety of additional technologies including alternative polypeptide-based technologies, such as fusions of complementarity determining regions as outlined in Qui et al., Nature Biotechnology, 25(8) 921-929 (2007), as well as nucleic acid-based technologies, such as the RNA aptamer technologies described in U.S. Pat. Nos. 5,789,157; 5,864,026; 5,712,375; 5,763,566; 6,013,443; 6,376,474; 6,613,526; 6,114,120; 6,261,774; and 6,387,620; all of which are hereby incorporated by reference, could be used in the context of the instant invention.

### CAR-T Cells

The present invention also provides chimeric antigen receptors (CARs) comprising an antigen binding domain of the antibodies of the present invention. Typically, said chimeric antigen receptor comprises at least one VH and/or VL sequence of the antibody of the present invention. The chimeric antigen receptor of the present invention also comprises an extracellular hinge domain, a transmembrane domain, and an intracellular T cell signaling domain. CAR-T cells have already been used in CTCL (see for example Scarfò I et al., 2019.

Thus, the present invention relates to an anti-ICOS CAR-T cells for use in the treatment of a cutaneous T-cell lymphomas (CTCL) and/or a T_{FH} derived lymphoma in a subject in need thereof.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the invention provides CARs comprising an antigen-binding domain comprising, consisting of, or consisting essentially of, a single chain variable fragment (scFv) of the antibodies of the invention. In some embodiments, the antigen binding domain comprises a linker peptide. The linker peptide may be positioned between the light chain variable region and the heavy chain variable region.

In some embodiments, the CAR comprises an extracellular hinge domain, a transmembrane domain, and an intracellular T cell signaling domain selected from the group consisting of CD28, 4-1BB, and CD3ζ intracellular domains. CD28 is a T cell marker important in T cell co-stimulation. 4-1BB transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs).

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

The invention also provides a nucleic acid encoding for a chimeric antigen receptor of the present invention. In some embodiments, the nucleic acid is incorporated in a vector as such as described above.

The present invention also provides a host cell comprising a nucleic acid encoding for a chimeric antigen receptor of the present invention. While the host cell can be of any cell type, can originate from any type of tissue, and can be of any developmental stage; the host cell is a T cell, e.g. isolated from peripheral blood lymphocytes (PBL) or peripheral blood mononuclear cells (PBMC). In some embodiments, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell can be any type of T cell and can be of any developmental stage, including but not limited to, CD4+/CD8+ double positive T cells, CD4+ helper T cells, e.g., Th2 cells, CD8+ T cells (e.g., cytotoxic T cells), tumor infiltrating cells, memory T cells, naive T cells, and the like. The T cell may be a CD8+ T cell or a CD4+ T cell.

The population of those T cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Adoptive immunotherapy of cancer refers to a therapeutic approach in which immune cells with an antitumor reactivity are administered to a tumor-bearing host, with the aim that the cells mediate either directly or indirectly, the regression of an established tumor. Transfusion of lymphocytes, particularly T lymphocytes, falls into this category. Currently, most adoptive immunotherapies are autolymphocyte therapies (ALT) directed to treatments using the patient's own immune cells. These therapies involve processing the patient's own lymphocytes to either enhance the immune cell mediated response or to recognize specific antigens or foreign substances in the body, including the cancer cells. The treatments are accomplished by removing the patient's lymphocytes and exposing these cells in vitro to biologics and drugs to activate the immune function of the cells. Once the autologous cells are activated, these ex vivo activated cells are reinfused into the patient to enhance the immune system to treat cancer. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 103/kg, preferably 5×103/kg; and as high as 107/kg, preferably 108/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

### Multispecific Antibodies

In some embodiments, the invention provides a multispecific antibody comprising a first antigen binding site from an antibody of the present invention molecule described herein above and at least one second antigen binding site.

Thus, the invention also relates to a multispecific antibody comprising a first antigen binding site from an antibody anti-ICOS of the invention and and at least one second antigen binding site for use in the treatment of a cutaneous T-cell lymphomas (CTCL) and/or a T_{FH} derived lymphoma in a subject in need thereof.

In some embodiments, the second antigen-binding site is used for recruiting a killing mechanism such as, for example, by binding an antigen on a human effector cell as a BiTE (Bispecific T-Cell engager) antibody which is a bispecific scFv2 directed against target antigen and CD3 on T cells described in U.S. Pat. No. 7,235,641, or by binding a cytotoxic agent or a second therapeutic agent. As used herein, the term “effector cell” refers to an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, mast cells and granulocytes, such as neutrophils, eosinophils and basophils. Some effector cells express specific Fc receptors (FcRs) and carry out specific immune functions. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs

In some embodiments, the second antigen-binding site binds to an antigen on a human B cell, such as, e.g., CD19, CD20, CD21, CD22, CD23, CD46, CD80, CD138 and HLA-DR.

In some embodiments, the second antigen-binding site binds a tissue-specific antigen, promoting localization of the bispecific antibody to a specific tissue.

In some embodiments, the second antigen-binding site binds to an antigen located on the same type of cell as the ICOS-expressing cell, typically a tumor-associated antigen (TAA), but has a binding specificity different from that of the first antigen-binding site. Such multi- or bispecific antibodies can enhance the specificity of the tumor cell binding and/or engage multiple effector pathways. Exemplary TAAs include carcinoembryonic antigen (CEA), prostate specific antigen (PSA), RAGE (renal antigen), a-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, Marti, MelanA, MUM-1, MUM-2, MUM-3, HLA-B7, Ep-CAM or a cancer-associated integrin, such as a503 integrin. Alternatively, the second antigen-binding site binds to a different epitope of [antigen]. The second antigen-binding site may alternatively bind an angiogenic factor or other cancer-associated growth factor, such as a vascular endothelial growth factor, a fibroblast growth factor, epidermal growth factor, angiogenin or a receptor of any of these, particularly receptors associated with cancer progression.

Exemplary formats for the multispecific antibody molecules of the invention include, but are not limited to (i) two antibodies cross-linked by chemical heteroconjugation, one with a specificity to ICOS and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is the antibody of the present invention. a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such asymmetrical mutations, optionally wherein one or both Fc-regions are of the IgG1 isotype.

In some embodiments, the first Fc region has an amino acid substitution at a position selected from the group consisting of: 366, 368, 370, 399, 405, 407 and 409, and the second Fc region has an amino acid substitution at a position selected from the group consisting of 366, 368, 370, 399, 405, 407 and 409, and wherein the first and second Fc regions are not substituted in the same positions.

In some embodiments, the first Fc region has an amino acid substitution at position 405, and said second Fc region has an amino acid substitution at a position selected from the group consisting of: 366, 368, 370, 399, 407 and 409, optionally 409.

In some embodiments, the first Fc region has an amino acid substitution at position 409, and said second Fc region has an amino acid substitution at a position selected from the group consisting of: 366, 368, 370, 399, 405, and 407, optionally 405 or 368.

In some embodiments, both the first and second Fc regions are of the IgG1 isotype, with the first Fc region having a Leu at position 405, and the second Fc region having an Arg at position 409.

### Immunoconjugates

An antibody of the invention can be conjugated with a detectable label to form an anti-ICOS immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below. The detectable label can be a radioisotope that is detected by autoradiography. Isotopes that are particularly useful for the purpose of the invention are 3H, 125I, 131I, 35S and 14C.

Anti-ICOS immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-ICOS immunoconjugates can be detectably labeled by coupling an antibody to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-ICOS immunoconjugates of the invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-ICOS immunoconjugates can be detectably labeled by linking an anti-[antigen] antibody to an enzyme. When the anti-ICOS-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include beta-galactosidase, glucose oxidase, peroxidase and alkaline phosphatase.

Those of skill in the art will know of other suitable labels which can be employed in accordance with the invention. The binding of marker moieties to anti-[antigen] monoclonal antibodies can be accomplished using standard techniques known to the art. Typical methodology in this regard is described by Kennedy et al., Clin. Chim. Acta 70:1, 1976; Schurs et al., Clin. Chim. Acta 81:1, 1977; Shih et al., Int'l J. Cancer 46:1101, 1990; Stein et al., Cancer Res. 50:1330, 1990; and Coligan, supra.

Moreover, the convenience and versatility of immunochemical detection can be enhanced by using anti-ICOS monoclonal antibodies that have been conjugated with avidin, streptavidin, and biotin. (See, e.g., Wilchek et al. (eds.), “Avidin-Biotin Technology,” Methods In Enzymology (Vol. 184) (Academic Press 1990); Bayer et al., “Immunochemical Applications of Avidin-Biotin Technology,” in Methods In Molecular Biology (Vol. 10) 149-162 (Manson, ed., The Humana Press, Inc. 1992).)

Methods for performing immunoassays are well-established. (See, e.g., Cook and Self, “Monoclonal Antibodies in Diagnostic Immunoassays,” in Monoclonal Antibodies: Production, Engineering, and Clinical Application 180-208 (Ritter and Ladyman, eds., Cambridge University Press 1995); Perry, “The Role of Monoclonal Antibodies in the Advancement of Immunoassay Technology,” in Monoclonal Antibodies: Principles and Applications 107-120 (Birch and Lennox, eds., Wiley-Liss, Inc. 1995); Diamandis, Immunoassay (Academic Press, Inc. 1996).)

In some embodiments, the antibody of the present invention is conjugated to a therapeutic moiety, i.e. a drug. The therapeutic moiety can be, e.g., a cytotoxin, a cytotoxic moiety, a chemotherapeutic agent, a cytokine, an immunosuppressant, an immune stimulator, a lytic peptide, or a radioisotope. Such conjugates are referred to herein as an “antibody-drug conjugates” or “ADCs”.

Thus, the invention also relates to an anti-ICOS antibody-drug conjugates (ADC) for use in the treatment of a cutaneous T-cell lymphomas (CTCL) and/or a T_{FH} derived lymphoma in a subject in need thereof.

In some embodiments, the antibodies of the invention are conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof, an antimitotic agent such as monomethyl auristatin E or F (MMAE or MMAF) or an analog or derivative thereof, dolastatin 10 or 15 or an analogue thereof, irinotecan or an analogue thereof, mitoxantrone; mithramycin; actinomycin D; 1-dehydrotestosterone; a glucocorticoid; procaine; tetracaine; lidocaine; propranolol; puromycin; calicheamicin or an analog or derivative thereof, an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof, an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, cytotoxic moiety an amatoxin selected in the group consisting in α-amanitin, β-amanitin, γ-amanitin, ε-amanitin, amanullin, amanullinic acid, amaninamide, amanin or aroamanullin.

In some embodiments, the antibodies of the invention are conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

In some embodiments, the antibodies of the invention are conjugated, e.g., as a fusion protein, to a lytic peptide such as CLIP, Magainin 2, mellitin, Cecropin and P18.

In some embodiments, the antibodies of the invention are conjugated to a cytokine, such as, e.g., IL-2, IL-4, IL-6, IL-7, IL-10, IL-12, IL-13, IL-15, IL-18, IL-23, IL-24, IL-27, IL-28a, IL-28b, IL-29, KGF, IFNa, IFN3, IFNy, GM-CSF, CD40L, Flt3 ligand, stem cell factor, ancestim, and TNFa.

In some embodiments, the antibodies of the invention are conjugated to a radioisotope or to a radioisotope-containing chelate. For example, the antibodies of the invention are can be conjugated to a chelator linker, e.g. DOTA, DTPA or tiuxetan, which allows for the antibody to be complexed with a radioisotope. The antibody may also or alternatively comprise or be conjugated to one or more radiolabeled amino acids or other radiolabeled molecules. Non-limiting examples of radioisotopes include 3H, 14C, 15N, 35S, 90Y, 99Tc, 125I, 131I, 186Re, 213Bi, 225Ac and 227Th. For therapeutic purposes, a radioisotope emitting beta- or alpha-particle radiation can be used, e.g., 1311, 90Y, 211At, 212Bi, 67Cu, 186Re, 188Re, and 212Pb.

In certain embodiments, an antibody-drug conjugate according to the invention comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, the antibodies of the invention are conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, beta-lactamase, beta-glucosidase, nitroreductase and carboxypeptidase A.

Typically, the antibody-drug conjugate of the invention comprises a linker unit between the drug unit and the antibody unit. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123).

Most typical are peptidyl linkers that are cleavable by enzymes that are present in 191P4D12-expressing cells. Examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values.

Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.) In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation.

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody-drug conjugate compound, are cleaved when the antibody-drug conjugate compound is present in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

Techniques for conjugating molecules to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

### Therapeutic Uses

As described above, the present invention relates to an anti-ICOS antibody for use in the treatment of a cutaneous T-cell lymphomas (CTCL) and/or a T_{FH} derived lymphoma in a subject in need thereof.

In another particular embodiment, the invention relates to an anti-ICOS antibody for use in the treatment of metastasis induced by a cutaneous T-cell lymphomas (CTCL) or a T_{FH} derived lymphoma in a subject in need thereof.

In each of the embodiments of the treatment methods described above, the anti-ICOS antibody or anti-ICOS antibody-drug conjugate (ADC) is delivered in a manner consistent with conventional methodologies associated with management of the disease or disorder for which treatment is sought. In accordance with the disclosure herein, an effective amount of the antibody or antibody-drug conjugate is administered to a patient in need of such treatment for a time and under conditions sufficient to prevent or treat the disease or disorder.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” or “effective amount” refers to an amount effective, at dosages and for periods of time necessary, to achieve a desired therapeutic result. A therapeutically effective amount of the antibody of the present invention may vary according to factors such as the disease state, age, sex, and weight of the individual, and the ability of the antibody of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present invention depend on the disease or condition to be treated and may be determined by the persons skilled in the art. A physician having ordinary skill in the art may readily determine and prescribe the effective amount of the pharmaceutical composition required. For example, the physician could start doses of the antibody of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. Alternatively, this property of a composition may be evaluated by examining the ability of the compound to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, one object of the present invention relates to a method of treating a cutaneous T-cell lymphomas (CTCL) and/or a T_{FH} derived lymphoma or metastasis induced by CTCL or T_{FH} derived lymphoma in a subject in need thereof comprising administering to the subject a therapeutically effective amount of the antibody of the present invention.

In a particular embodiment, the present invention relates to a method of treating a CTCL (skin and blood involvement) in a subject in need thereof comprising administering to the subject a therapeutically effective amount of the antibody of the present invention.

In certain embodiments, an anti-ICOS antibody or antibody-drug conjugate (ADC) is used in combination with a second agent for treatment of a disease or disorder. When used for treating a cutaneous T-cell lymphomas (CTCL) and/or a T_{FH} derived lymphoma or a metastasis induced by CTCL or T_{FH} derived lymphoma, an anti-ICOS antibody or ADC of the invention may be used in combination with conventional cancer therapies such as, e.g., surgery, radiotherapy, chemotherapy, or combinations thereof.

The present invention also provides for therapeutic applications where an antibody of the present invention is used in combination with at least one further therapeutic agent, e.g. for treating cancers and metastatic cancers. Such administration may be simultaneous, separate or sequential. For simultaneous administration the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In some embodiments, the antibody of the present invention is used in combination with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the antibody of the present invention is used in combination with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the antibody of the present invention is used in combination with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC2+ and/or CD8+ T cells (for instance T cells expanded with tumor-specific antigens and/or genetic enhancements), antibody-expressing B cells or other antibody-producing or -presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions.

In some embodiments, the antibody of the present invention is used in combination with radiotherapy. Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the antibody of the present invention is used in combination with an antibody that is specific for a costimulatory molecule. Examples of antibodies that are specific for a costimulatory molecule include but are not limited to anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies.

In some embodiments, the second agent is an agent that induces, via ADCC, the death of a cell expressing an antigen to which the second agent binds. In some embodiments, the agent is an antibody (e.g. of IgG1 or IgG3 isotype) whose mode of action involves induction of ADCC toward a cell to which the antibody binds. NK cells have an important role in inducing ADCC and increased reactivity of NK cells can be directed to target cells through use of such a second agent. In some embodiments, the second agent is an antibody specific for a cell surface antigens, e.g., membrane antigens. In some embodiments, the second antibody is specific for a tumor antigen as described above (e.g., molecules specifically expressed by tumor cells), such as CD20, CD52, ErbB2 (or HER2/Neu), CD33, CD22, CD25, MUC-1, CEA, KDR, aVP3, etc., particularly lymphoma antigens (e.g., CD20). Accordingly, the present invention also provides methods to enhance the anti-tumor effect of monoclonal antibodies directed against tumor antigen(s). In the methods of the invention, ADCC function is specifically augmented, which in turn enhances target cell killing, by sequential administration of an antibody directed against one or more tumor antigens, and an antibody of the present invention.

Accordingly, a further object relates to a method of enhancing NK cell antibody-dependent cellular cytotoxicity (ADCC) of an antibody in a subject in need thereof comprising administering to the subject the antibody, and administering to the subject an antibody of the present invention.

A further object of the present invention relates to a method of treating a cutaneous T-cell lymphomas (CTCL) and/or a T_{FH} derived lymphoma in a subject in need thereof comprising administering to the subject a first antibody selective for a cancer cell antigen, and administering to the subject an antibody of the present invention.

A number of antibodies are currently in clinical use for the treatment of cancer, and others are in varying stages of clinical development. Antibodies of interest for the methods of the invention act through ADCC, and are typically selective for tumor cells, although one of skill in the art will recognize that some clinically useful antibodies do act on non-tumor cells, e.g. CD20. There are a number of antigens and corresponding monoclonal antibodies for the treatment of B cell malignancies. One popular target antigen is CD20, which is found on B cell malignancies. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Monoclonal antibodies targeting CD20, also include tositumomab and ibritumomab. Monoclonal antibodies useful in the methods of the invention, which have been used in solid tumors, include without limitation edrecolomab and trastuzumab (herceptin). Edrecolomab targets the 17-1 A antigen seen in colon and rectal cancer, and has been approved for use in Europe for these indications. Its antitumor effects are mediated through ADCC, CDC, and the induction of an anti-idiotypic network. Trastuzumab targets the HER-2/neu antigen. This antigen is seen on 25% to 35% of breast cancers. Trastuzumab is thought to work in a variety of ways: downregulation of HER-2 receptor expression, inhibition of proliferation of human tumor cells that overexpress HER-2 protein, enhancing immune recruitment and ADCC against tumor cells that overexpress HER-2 protein, and downregulation of angiogenesis factors. Alemtuzumab (Campath) is used in the treatment of chronic lymphocytic leukemia; colon cancer and lung cancer; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

### Pharmaceutical Compositions

Typically, the antibodies of the present invention are administered to the subject in the form of a pharmaceutical composition which comprises a pharmaceutically acceptable carrier.

Thus, the invention also relates to a pharmaceutical composition comprising an anti-ICOS antibody for use in the treatment of a cutaneous T-cell lymphomas (CTCL) and/or a TFH derived lymphoma in a subject in need thereof.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m2 and 500 mg/m2. However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-myosin 18A antibody of the invention.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MHLVs)). MLVs generally have diameters of from 25 nm to 4 μm. Sonication of MHLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the following figures and examples. However, these examples and figures should not be interpreted in any way as limiting the scope of the present invention.

### FIGURES

FIG. 1: Anti-ICOS ADCs have a specific in vitro efficacy in ICOS-expressing cell lines. A. Anti-ICOS ADCs have a specific in vitro efficacy in ICOS-expressing cell lines. (A-E) Percentage of cell viability in increasing ADC concentrations, assessed with alamarBlue™ (mean of 16 replicates), on MyLa cells (A), MJ cells (B), HUT78 cells (C), Jurkat cells (D) and Jurkat-ICOS cells (E). Anti-HER2 ADCs were used as negative control, whereas anti-CD30 ADCs (BV) were positive control. ***: p<0.001; **: p=0.001-0.01; *: p=0.01-0.05; ns: not significant.

FIG. 2. Evaluation of the in vivo efficacy of anti-ICOS-MMAE ADCs in a mouse xenograft model with MyLa cells. (A) Twenty-one mice were engrafted with 8.106 MyLa cells each, which were subcutaneously injected with 200 μL of PBS and no basement membrane matrix. Mice were then randomly assigned to three groups were monitored for tumor volume after two treatments administered 4 days apart (D10 and D14 after engraftment) of either anti-HER-2, anti-CD30, or anti-ICOS ADCs. (B) Overall survival curves (Kaplan-Meier) comparing the effect of anti-ICOS and anti-CD30 ADCs. The difference between the two curves is significant (p=0.0006). (C-E) Detection of the development of bioluminescent MyLa metastases in 26 mice assigned to three groups and treated with either anti-HER2, anti-CD30, or anti-ICOS ADCs, in lungs (C), spleen (D) and liver (E).

FIG. 3. In vivo efficacy of anti-ICOS-MMAE ADCs on ICOS+ PDXs. (A-C) Fourteen mice were engrafted with 5.105 cells of PDXs from patients with SS and assigned in two groups (anti-ICOS-MMAE ADC group and the anti-HER2 ADC control group). Both treatments were injected at D55, D58, D62 and D65 at the dose of 3 mg/kg IV. Mice were then sacrificed at D69 and organs were removed, dissociated and analyzed by flow cytometry (A: in the blood; B: in the bone marrow; C: in the spleen). (D) Thirty mice were engrafted with 5.105 cells of PDXs from patients with AITL, and were assigned in three groups of 10 mice. Treatment began at D22, when the earliest blasts were detected in the mice' blood (approximately 0.2 blasts/μl). Anti-ICOS ADC and saline serum (NaC 0.96) were injected at D22, D25, D38 and D43, at the dose of 3 mg/kgs IV. Vincristine were administered at D22, D29 and D38 at 0.25 mg/kgs IP. *: p=0.01 to 0.05. ***: p0.001.

FIG. 4. MAB-Zap assay allows the identification of ICOS clones that would be the best candidates for the development of anti-ICOS ADCs. (A) Schematic representation of the way MAB-Zap operates. (B) Percentage of cell viability in increasing ADC 10 concentrations on MJ, assessed with AlamarBllue™. Note that anti-ICOS 53.3-MMAE and anti-ICOS 92.17-MMAE are more effective than anti-ICOS 314.8-MMAE.

TABLE 3
Summary table of IC50 values expressed in ng/ml of all the ADCs

| | ICOS-MMAE | ICOS-PBD | CD30-MMAE |
|---|---|---|---|
| Myla | 8.2 | 1.2 | 30.6 |
| MJ | 36.2 | 0.8 | 6.5 |
| HUT78 | | 9 | 251.9 |
| Jurkat | | | 733 |
| Jurkat-ICOS | 6.7 | 0.7 | 128 |

TABLE 4
Efficacy of each anti-ICOS mAbs, expressec as IC50, to act
as ADCs with MAB-Zap assay.

| | MyLa | MJ |
|---|---|---|
| | Efficacy with | Efficacy with |
| ICOS clones | MAB-Zap (IC50) | MAB-Zap (IC50) |
| 314.8 | 0.05 | 0.84 |
| 92.17 | 0.19 | 0.46 |
| 53.3 | 0.1 | 0.27 |
| 293.1 | 0.09 | 0.23 |
| 88.2 | 0.12 | 0.32 |
| 279.1 | >1000 | 0.38 |
| 145.1 | >1000 | >1000 |
| 121.4 | >1000 | >1000 |

### EXAMPLE

### Example 1: Use of an Anti-ICOS Antibody-Drug Conjugates (ADC)

Material & Methods

Study Design and Population

We conducted a prospective multicenter study between November 2017 and October 2018. Patients were >18 years old and signed written informed consent forms prior to the initiation of any procedure related to the study. The diagnosis of CTCL was carried out by a clinician and a pathologist, both members of the French Cutaneous Lymphoma Group (GFELC: Groupe Frangais d'Etude des Lymphomes Cutanes). We characterized each patient according to the 2018 WHO-EORTC diagnosis and classification criteria.25 We then performed clinical staging according to the revised staging system for CTCL, based on the tumor-node-metastasis-blood (TNMB) classification system.26 To confirm a diagnosis of SS, the patient had to meet the criteria of group B2 of the TNMB classification. For functional tests, patients with SS were included either at initial diagnosis or at clinical and biological relapse (B2 criteria). We excluded patients undergoing treatment with immunotherapy or in a therapeutic trial.

Skin samples from 52 patients with CTCL at diagnosis (38 patients) or in relapse (14 patients) were obtained by 4-mm punch biopsy under local anesthesia then fixed with formaldehyde and embedded in paraffin. Blood samples from 13 patients with SS consisted of 15 mL of whole blood in EDTA tubes. Skin samples from 12 patients with B-cell lymphoma, 14 with CD30+ lymphoproliferative disease (LPD) (cutaneous anaplastic large cell lymphoma and lymphomatoid papulosis), 12 with PCSMLPD and 13 with AITL were used as control. The clinical characteristics of patients with CTCL and controls are summarized in Supplementary Table S1. The healthy volunteers were blood donors at the Etablissement Frangais du Sang (EFS).

All patient tissue collection and research use adhered to protocols approved by the Institutional Review and Privacy Boards at Institut Paoli-Calmettes (ICOS-LYMPH-IPC2018003), Saint-Louis Hospital, and the Henri-Mondor Hospital, in accordance with the Declaration of Helsinki.

Generation of mAbs

For the generation of anti-ICOS ADCs, a purified murine anti-ICOS antibody generated in our laboratory 27 was sent to Levena Biopharma and Concortis Biotherapeutics (San Diego, Calif., USA) for coupling to MMAE and pyrrolobenzodiazepine (PBD).

BV (anti-CD30-MMAE) and ado-trastuzumab emtansine (anti-HER2-MMAE) were provided by our hospital pharmacy.

Cell Culture

We used three CTCL cell lines: MyLa (from: Pr N. Ortonne, Department of Pathology, Henri-Mondor Hospital, Creteil, France), MJ (from: American Type Culture Collection [ATCC], VA, USA) and HUT78 (from: ATCC). Myla and MJ are MF cell lines, while HUT78 is a SS cell line. MyLa and HUT78 cells were cultured in RPMI 1640 medium (Life Technologies) supplemented with 10% fetal calf serum (FCS), 2% L-glutamine, 1% pyruvate; MJ in Iscove's modified Dulbecco's medium (IMDM) (Life Technologies) supplemented with 20% FCS. The diffuse large B-cell lymphoma (Daudi, ATCC CCL-213) and T-cell leukemia (Jurkat, ATCC TIB-152) cell lines were also purchased from ATCC and were cultured in the same way as MyLa and HUT78 cells. The Jurkat cell line that was transfected to express the ICOS receptor was named Jurkat-ICOS. The MyLa cell line transfected to express luciferase (infection with lentivirus vector expressing LUC2) was named MyLa-Luciferase.

Patient-derived xenografts (PDXs) of AITL (DFTL 78024V1) and SS (DFTL 90501V3) were obtained from the Dana-Farber Cancer Institute, Boston (MA, USA) 28.

Flow Cytometry and Immunochemistry

We used rabbit anti-ICOS antibodies (rabbit polyconal antibody from Spring Biosciences [Abcam, Cambridge, UK] for immunohistochemistry, and the SP98 rabbit monoclonal antibody, Spring Biosciences, with anti-rabbit Alexa488 secondary antibodies), as well as mouse antibodies to PD-1 (NAT105, Abcam), CD4 (4B12, Novocastra) (Leica Biosystems, Wetzlar, Germany), CD8 (C8/144B, Dako) (Agilent Technologies, Santa Clara, Calif., USA), and FoxP3 (236A/E7, Abcam) for fluorescent multiplex stainings using anti-mouse Texas red secondary antibodies and DAPI for nuclear stainings. All staining experiments were done on 3 m thick sections from formalin fixed paraffin embedded skin and node biopsies, either manually or using the Bond Max device (Leica Microsystems). The expression of ICOS and all other markers was scored semi-quantitatively and divided into four categories, based on the proportion of positive cells within the tumoral T-cell infiltrate (0: no staining, low expression: <5%, moderate expression: 5-50%, high expression: >50%).

For flow cytometry and functional tests, we used anti-ICOS 314.8 antibodies generated in our laboratory (for details, see Le et a127). Other antibodies were purchased from Beckman Coulter (BC) (Brea, Calif., USA), Becton-Dickinson (BD) (Franklin Lakes, N.J., USA), Miltenyi Biotech (Bergisch Gladbach, Germany), and eBioscience (San Diego, Calif., USA): CD45 KO (BC), CD3 percpCy5.5 (BD), CD4 Pacblue (BD), CD7 FITC (BD), CD26 APC (Miltenyi), CD14 APCH7 (BD), CD158e/k PE Vio770 (Miltenyi), CD52 PE (Miltenyi), CD56 APC-Vio770 (Miltenyi), CD19 APC (BD), CD20 PE (BC), CD25 PE-Cf 594 (BD), and FoxP3 FITC (eBioscience).

Flow cytometric analyses were performed on a FACS Canto II (BD Biosciences, San Jose, Calif., USA) cytometer. The raw data generated were analyzed with the DIVA FACS Canto II software version 8.0.1.

Measurement of Cell Line Viability in the Presence of ADCs

Cell viability was measured with alamarBlue™ (Biosource, Carlsbad, Calif., USA). After 4 to 5 days of cell exposure to ADCs, alamarBlue™ was added. After 4 hours of incubation at 37° C., fluorescence was measured by a luminometer (OPTIMA, BMG Labtech) at a wavelength of 560 nm, as recommended by the manufacturer.

Animals and Xenograft Models

All experiments were done in agreement with the French Guidelines for animal handling, the ARRIVE guidelines and approved by local ethics committee (Agreement no. APAFIS #6069-2016071216263470 v3).

Non-obese diabetic severe combined immunodeficiency gamma (NSG/NOD.Cg-Prkdcscid Il2rgtm1Wjl/SzJ) male mice of 6-8 weeks of age were used for mouse studies and were obtained from Charles Rivers (l'Arbresle, France). Mice were housed under sterile conditions with sterilized food and water provided ad libitum and were maintained on a 12-h light and 12-h dark cycle and under temperature and humidity control. Cages contained an enriched environment with bedding material.

Mice were given subcutaneous injections of 8 million Myla or MylaLuc cells in PBS. Tumor growth was monitored by measuring with a digital caliper and calculating tumor volume (length×width2×7π/6). When tumors reached an average size close to 100 mm3, mice were randomized (n=7 per group) and used to determine the treatment response. Treatments with ADCs were injected intravenously into the caudal vein. BV and anti-ICOS ADCs were administered at the same dose (3 mg/kg) and ado-trastuzumab emtansine at 10 mg/kg. Bioluminescence analysis was performed using a PhotonIMAGER (Biospace Lab, Nesles-la-Vallée, France) following addition of endotoxin-free luciferin (30 mg/kg). After completion of the analysis, mice autopsies were performed, and organ luminescence was assessed. Daily monitoring of mice for symptoms of disease (tumor volume >1500 mm3, significant weight loss, ruffled coat, hunched back, weakness, and reduced mobility) determined the time of killing for injected animals with signs of distress. Survival curves were estimated by the Kaplan-Meier method and compared using the log-rank test.

To explore the efficiency of ADC treatments on lymphoma progression, we utilized PDXs of AITL (DFTL 78024V1) and SS (DFTL 90501V3). For each PDX, 100,000-500,000 cells from the PDXs were injected intravenously into the caudal vein of NSG mice without prior in vitro culture. When mice were engrafted (hCD45⁺ cells detected in peripheral blood by flow cytometry), NSG mice were treated in the same manner as previously described.

Statistical Analysis

All data were analyzed with the GraphPad Prism program (GraphPad Software, San Diego, Calif., USA). An unpaired non-parametric Student's t-test with level of significance set at p<0.05 was used to compare the in vitro efficacy of the antibody of interest and its control. The grouped efficacy analyses were performed with a two-way analysis of variance (ANOVA) test. IC50 (median inhibitory dose) was calculated with non-linear regression. The in vivo survival curves were compared with the log-rank test (Kaplan-Meier).

Results

ICOS is Widely Expressed by Malignant Cells in the Skin of Patients with MF and SS

We used immunohistochemistry to study ICOS expression in skin biopsies of 52 patients with CTCL at diagnosis (38 patients) or in relapse (14 patients). In 5 patients with SS, we also analyzed concomitant core biopsies from histologically proven involved nodes with tumor T-cell invasion at histological evaluation (pN3). We measured the ICOS expression of the CD3⁺ tumoral T-cell population which was characterized morphologically (nuclear atypias) and phenotypically (pan-T-cell antigen loss amongst CD2, CD5, CD7; PD-1 expression for SS samples; CD30 expression for primary cutaneous CD30⁺ T-cell lymphoproliferative disorders [LPD]).

Atypical lymphocytic infiltrates in 61% of 23 patients with early-stage MF (stages IA to IIA, without large cell transformation) showed moderate to high ICOS expression. Tumoral cells from 75% of the 12 patients with transformed MF had moderate to high expression of ICOS. Finally, ICOS was highly expressed by 15/17 (88%) of the skin biopsies of patients with SS (data not shown). As expected, ICOS was poorly expressed in B-cell lymphoma and widely expressed in PCSMLPD and AITL tumoral infiltrates. Interestingly, tumoral cells of CD30⁺ LPD exhibited a low expression of ICOS. Moreover, ICOS was expressed by atypical lymphocytes in all the five nodes with SS involvement, being highly expressed in four of them. Therefore, ICOS expression increases with the progression of the disease and becomes widely expressed in SS, both in the skin and nodes.

Double staining experiments were performed in both skin and lymph node samples from these five patients to further characterize ICOS expression by neoplastic T-cells and by the microenvironment (data not shown). We observed that most atypical CD4⁺ T-cells (>50%) expressed ICOS, as well as most PD-1⁺ atypical cells, except for one patient with a low to moderate ICOS expression. In the latter, all ICOS⁻ lymphocytes co-expressed PD-1. A high PD-1 expression was found in all skin and node sample, and ICOS⁺PD-1⁻ lymphocytes appeared to be absent or very rare (<5%). Only very few (<5%) ICOS⁺CD8⁺ T-cells could be identified in the tumor microenvironment in 3 node samples. Few to moderate amounts of CD4⁺ T-cells appeared to be ICOS− in the skin and node samples. A low proportion of FoxP3⁺ Tregs lymphocytes were identified in 3 cases, both in the skin and lymph nodes for two and only in the node for one. A low to moderate proportion of them expressed ICOS (data not shown). Thus, ICOS expression appears mainly restricted to neoplastic CD4⁺ T-cells, with rare ICOS⁺CD8⁺ T-cells or FoxP3⁺ Tregs in the tumor micro-environment.

ICOS is Widely Expressed by Malignant Cells in the Blood of Patients with SS

ICOS expression by circulating malignant cells was then evaluated using flow cytometry. To ensure the most specific selection of Sézary cells, we considered CD4⁺KIR3DL2⁺ T-cells with loss of either CD7 or CD26 to be malignant cells. Data shows the distribution of lymphocyte populations in 13 patients compared to 12 healthy volunteers. In patients, the median percentage of malignant CD4⁺ T-cells (Sézary cells) among all lymphoid cells was 53.1% (35.9-71), meaning that 64% of all CD4⁺ T-cells in patients were malignant cells. Tregs (CD4⁺CD25⁺FoxP3⁺) accounted for 2% of all lymphocytes, i.e. 4.3% of non-tumoral lymphocytes; this was 3.4% in healthy donors. In addition, NK lymphocytes made up 2.4% of all lymphocytes in patients (5% of non-tumoral lymphocytes), compared to 5.5% in healthy donors. NK lymphocytes did not express ICOS (data not shown).

Expression of ICOS by circulating tumor cells was found in all patients. The expression was strong: 69±7.3% of tumor cells expressed ICOS versus 38.8±7.1% of non-tumoral CD4⁺ cells in patients (p<0.009; 95% confidence interval [CI95%]: 8.654-51.55); and 31±3.2% of CD4+ cells in healthy volunteers (p<0.0001; CI95%: 20.29-46.34) (data not shown). In patients, 14.4±2.7% of Foxp3⁺CD25⁺CD4⁺ Tregs expressed ICOS, compared to 5.6±1.2% in healthy volunteers (p=0.04) (data not shown).

Anti-ICOS ADCs Mediate Killing of MyLa, MJ and HUT78 Cell Lines

We first tested anti-ICOS ADCs on MF (MyLa and MJ) and SS (HUT78) cell lines to ensure their functionality. ICOS expression was strong on MyLa (MFI ratio=143.9) and MJ (MFI ratio=96) but low on HUT78 (MFI ratio=4.5). CD30 was strongly expressed on all 3 cell lines (data not shown).

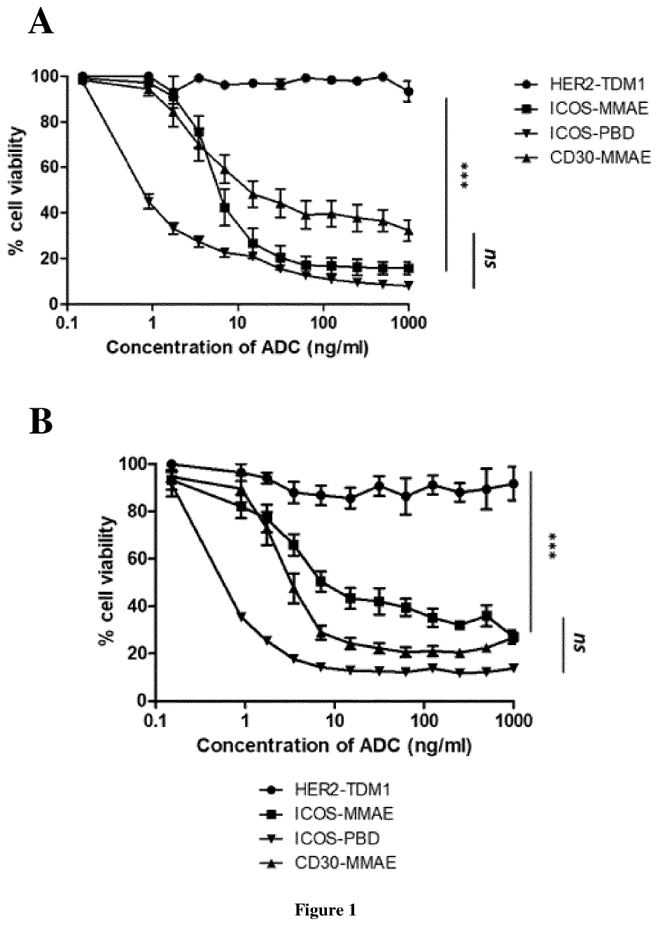

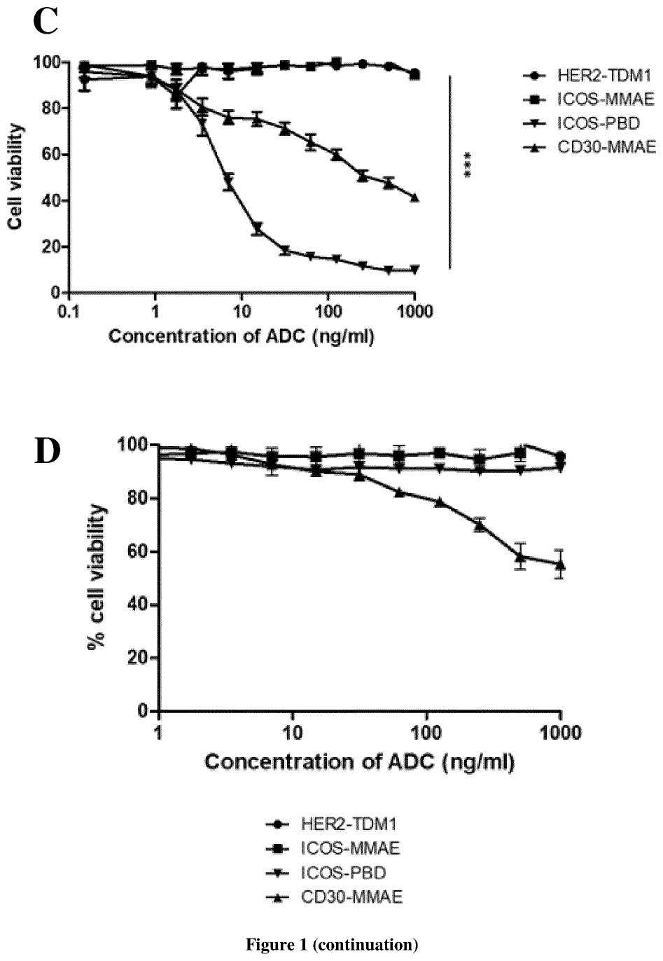

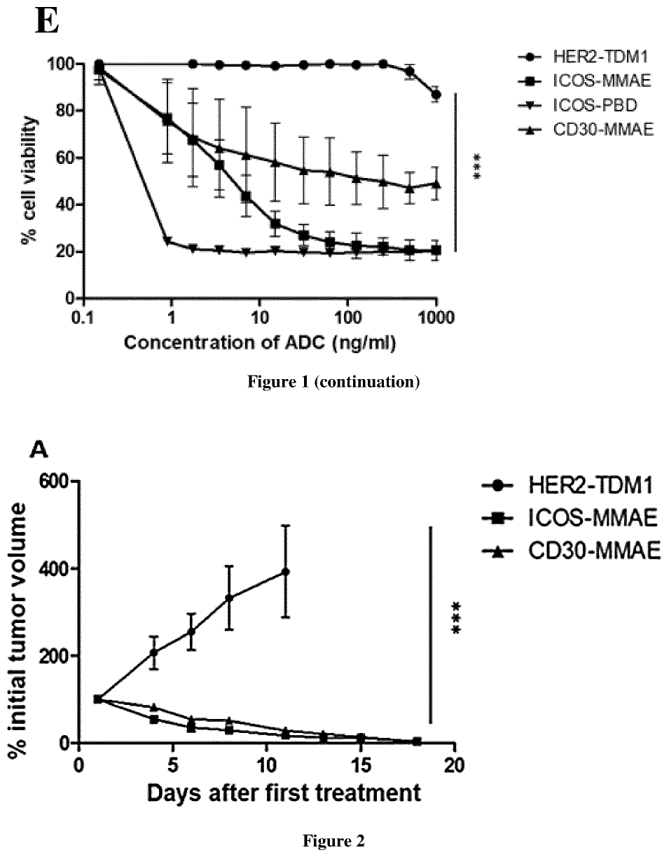

We observed a significant dose-dependent decrease in cell viability in the presence of anti-ICOS-MMAE ADCs in the MyLa and MJ cell lines (FIGS. 1A-B). In the MyLa cell line, anti-ICOS-MMAE ADCs had a better but not statistically significant different IC50 than BV (respectively 8.2 ng/ml and 30.6 ng/ml). In MJ cells, the anti-ICOS-MMAE ADCs tended to be less effective than BV. This difference could be explained by the fact that anti-ICOS mAbs 10 were internalized more in MyLa than in MJ cells, while the opposite occurred for anti-CD30 mAbs (data not shown).

In HUT78 cells, BV is less effective than in MyLa and MJ (IC50=251.9 ng/ml) and anti-ICOS-MMAE ADCs exhibit no activity (FIG. 1C). Indeed, HUT78 cell line displays resistance to MMAE, as IC50 of free-MMAE is respectively of 8.2e-007 μM and 0.001 μM in MyLa and HUT78 (data not shown). However, anti-ICOS-PBD ADCs mediate potent killing of the cells, suggesting that anti-ICOS ADCs coupled with a well-adapted drug could be effective even with low levels of ICOS expression.

Finally, we assessed the specificity of ADCs by testing the anti-ICOS ADCs on Jurkat and Jurkat-ICOS cells (FIGS. 1D-E). IC50 values of all the ADCs are summarized in Table 3.

In Vivo, Anti-ICOS-MMAE ADCs are Superior to BV in Terms of Overall Survival and Prevents the Development of Metastases

Mice subcutaneously engrafted with 8.106 MyLa cells were randomly assigned to three groups: an anti-ICOS-MMAE ADC group, BV group, anti-HER2 (ado-trastuzumab-emtansine) ADC group.

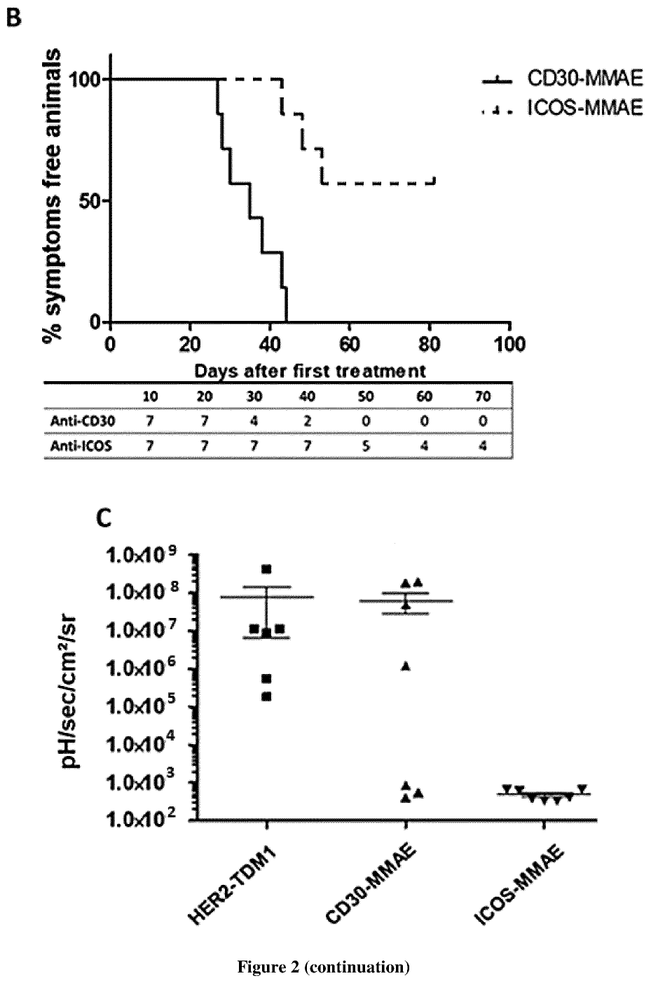

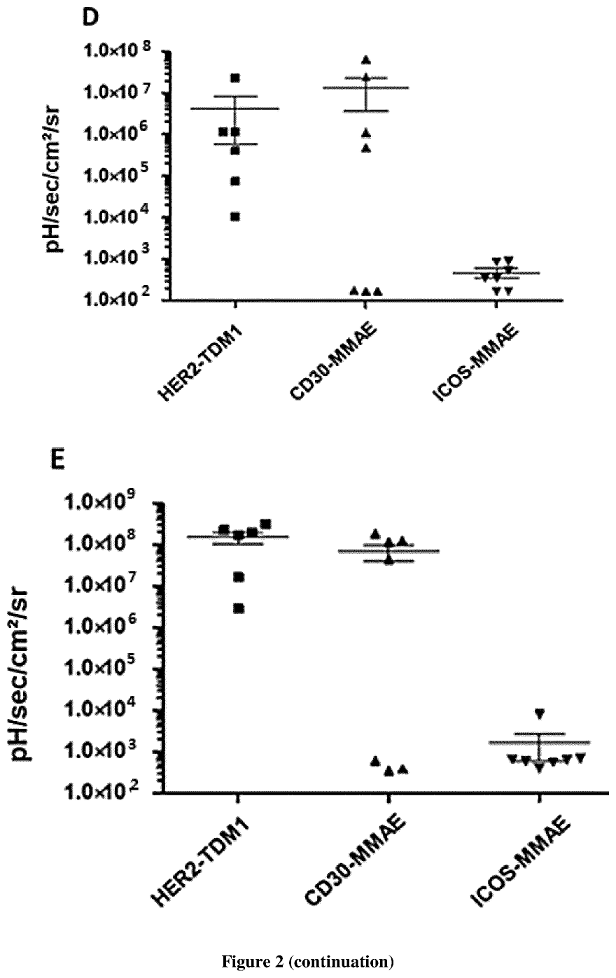

Mice treated with anti-HER2 ADCs died between day (D)10 and D12. A rapid decline in tumor volume occurred after treatment with anti-ICOS-MMAE ADCs or BV (FIG. 2A).

Subcutaneous tumor volumes were no longer noticeable from the fifteenth day after the first injection, with no significant difference between the two treatments. Tolerance was excellent, with no evidence of ADC toxicity in treated mice. Interestingly, anti-ICOS-MMAE ADCs provided a longer overall survival (OS) than BV (HR=15.2; CI95%: 3.2-71.1; p<0.0006) (FIG. 2B). The median survival in the BV group was 35 days and was not reached in the anti-ICOS ADC group.

In a second experiment, we aimed to monitor the development of metastases using MyLa-Luciferase cells. Twenty-seven mice were engrafted and treated under the same conditions as in the first experiment. On D25, 7 mice from each group were sacrificed and their organs were scanned with the luminometer to detect the presence of metastases. The other mice were maintained until D40 to detect in vivo the onset of subcutaneous recurrence. On D25, all mice in the anti-HER2 group had metastases in the lungs, liver, and spleen. In the BV group, around 50% of mice had at least one metastasis in one of these three organs. In the anti-ICOS-MMAE group, the organs did not exhibit significant bioluminescence (FIGS. 2C,D,E). On D40, subcutaneous recurrence was perceived in vivo in mice of the BV group, while mice in the anti-ICOS group were still in remission (data not shown).

Anti-ICOS-MMAE ADCs have a Potent In Vivo Efficacy in PDXs of ICOS+ Lymphomas

To improve the predictive value of our preclinical model, we assessed the efficacy of anti-ICOS-MMAE ADCs in ICOS⁺PDXs from patients with SS and AITL.

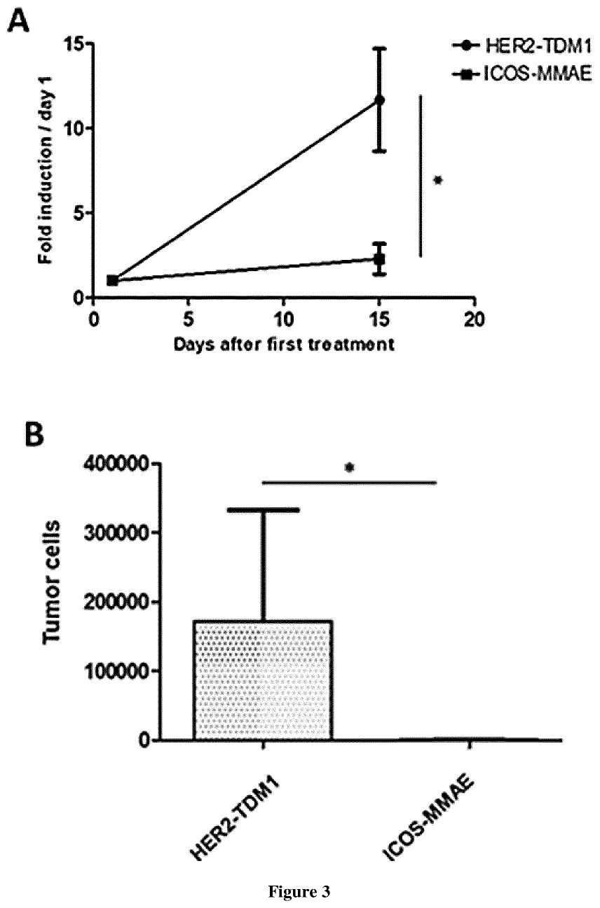

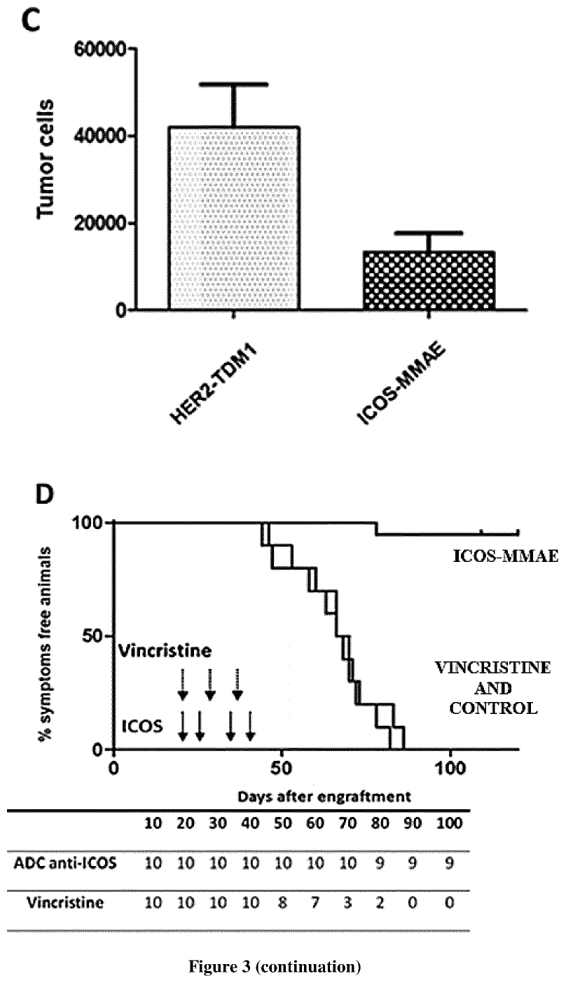

ICOS⁺PDXs from patients with SS were intravenously injected into fourteen NSG mice. On D40 after engraftment, we observed a brutal and rapid increase in the number of Sézary cells. We took blood samples from each mouse and quantified the number of circulating tumor cells to evenly distribute the living mice into two groups of 7 mice: the anti-ICOS-MMAE ADC group and the anti-HER2 ADC control group. Fifteen days after treatment, the mice were sacrificed, and we quantified the number of malignant cells in the blood and organs by flow cytometry. We observed a reduced number of tumor cells in the blood, bone marrow, and spleen of the anti-ICOS ADC group (FIG. 3A,B,C). Anti-ICOS ADCs here show a rapid and significant efficacy, suggesting that this therapeutic strategy could be used in patients with advanced SS.

In a second experiment, ICOS⁺PDXs from patients with AITL were intravenously 25 injected into NSG mice. We subsequently took blood samples to detect tumor cells by flow cytometry. The first tumor cells were detected on D21 after transplantation, so treatments began on D22. Mice were treated with anti-ICOS-MMAE ADCs, vincristine (positive control, with the same mode of action as MMAE), or saline solution (NaCl à.9%). Median survival in the negative control and vincristine group was D67 and D68, respectively. Median survival in the anti-ICOS group was not reached. The better survival of mice treated with anti-ICOS ADC compared to those receiving saline solution was highly significant (p<0.0001) (FIG. 3D). No evidence of ADC toxicity was observed in treated mice. On D120, the mice treated with anti-ICOS ADCs were in complete remission since no blasts were more detectable. (data not shown).

### Example 2: Use of Others Anti-ICOS Antibodies-Drug Conjugates (ADC)

Material & Methods

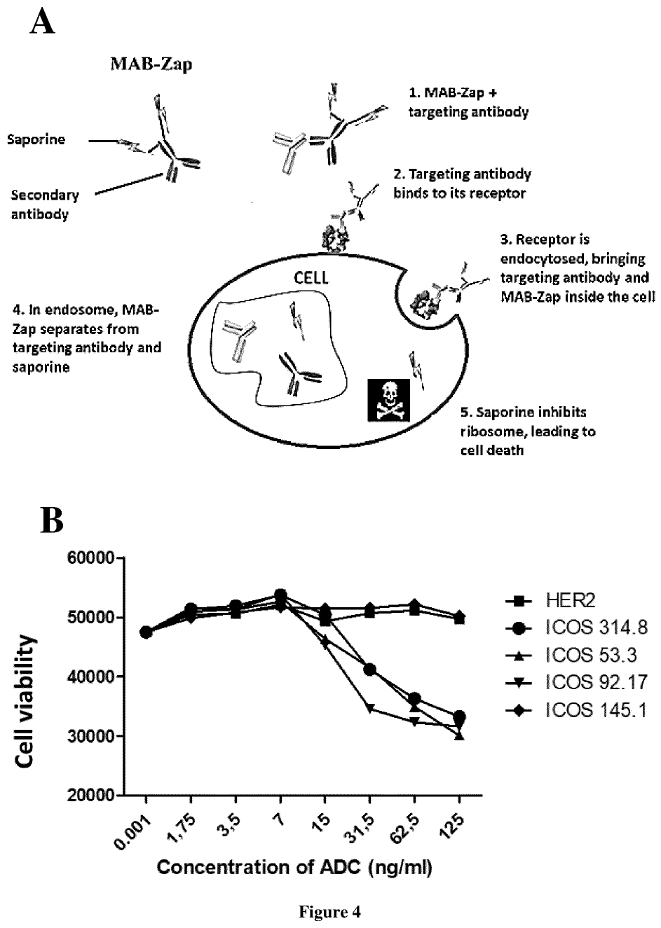

To assess the ability of different anti-ICOS antibodies to act as ADCs, we used MAB-Zap (Advanced Targeting System, San Diego, USA), which is a secondary anti-murine IgG antibody coupled to saporin, a ribosome inhibitor (FIG. 4A). The MAB-Zap recognizes the Fc fragment of our antibody of interest, then the MAB-Zap-Antibody complex binds to the surface antigen and is internalized. Saporin is released into the cytosol and inhibits the ribosome, stopping protein synthesis and resulting in cell death. The commercial kit also includes a negative control corresponding to serum polyclonal Ig, IgG-SAP, also coupled with saporin.

In 96-well round-bottomed plates, cells are exposed to purified antibodies at increasing concentrations from OnM to 40 nM. MAB-Zap is added at a concentration of 4.5 nM (manufacturer's recommendations), as well as IgG-SAP in the control wells. After 3 days incubation at 37° C., AlamarBlue is added to each well (10% of the total well volume) and the fluorescence is read with OPTIMA luminometer.

Results

We tested on MyLa and MJ the 9 following anti-ICOS mAbs: 314.8, 92.17, 53.3, 298.1, 88.2, 279.1, 145.1, 121.4. All these mAbs showed an ability to act as ADCs using the MAB-Zap assay except for 3 on MyLa (279.1, 145.1 and 121.4) and 2 on MJ (145.1 and 121.4). The efficacy of each mAbs, expressed as IC50, is shown in Table 4. To confirm these results, we coupled 53.3, 92.17 and 145.1 anti-ICOS mAbs to MMAE, and compared them to our first 314.8 anti-ICOS ADCs (FIG. 4B).

### REFERENCES

Throughout this application, various references describe the state of the art to which this invention pertains. The disclosures of these references are hereby incorporated by reference into the present disclosure.
1. Swerdlow S H, Campo E, Pileri S A, et al. The 2016 revision of the World Health Organization classification of lymphoid neoplasms. Blood. 2016; 127(20):2375-2390. doi:10.1182/blood-2016-01-643569
2. Jawed S I, Myskowski P L, Horwitz S, Moskowitz A, Querfeld C. Primary cutaneous T-cell lymphoma (mycosis fungoides and Sézary syndrome): part I Diagnosis: clinical and histopathologic features and new molecular and biologic markers. J Am Acad Dermatol. 2014; 70(2):205.e1-16; quiz 221-222. doi:10.1016/j.jaad.2013.07.049
3. Scarisbrick J J, Hodak E, Bagot M, et al. Blood classification and blood response criteria in mycosis fungoides and Sézary syndrome using flow cytometry: recommendations from the EORTC cutaneous lymphoma task force. Eur J Cancer Oxf Engl 1990. 2018; 93:47-56. doi:10.1016/j.ejca.2018.01.076
4. Roelens M, de Masson A, Ram-Wolff C, et al. Revisiting the initial diagnosis and blood staging of mycosis fungoides and Sézary syndrome with the KIR3DL2 marker. Br J Dermatol. September 2019. doi:10.1111/bjd.18481
5. Bahler D W, Hartung L, Hill S, Bowen G M, Vonderheid E C. CD158k/KIR3DL2 is a useful marker for identifying neoplastic T-cells in Sézary syndrome by flow cytometry. Cytometry B Clin Cytom. 2008; 74(3):156-162. doi:10.1002/cyto.b.20395
6. Ortonne N, Le Gouvello S, Mansour H, et al. CD158K/KIR3DL2 transcript detection in lesional skin of patients with erythroderma is a tool for the diagnosis of Sézary syndrome. J Invest Dermatol. 2008; 128(2):465-472. doi:10.1038/sj.jid.5701013
7. Samimi S, Benoit B, Evans K, et al. Increased programmed death-1 expression on CD4+ T cells in cutaneous T-cell lymphoma: implications for immune suppression. Arch Dermatol. 2010; 146(12):1382-1388. doi:10.1001/archdermatol.2010.200
8. Wada D A, Wilcox R A, Harrington S M, Kwon E D, Ansell S M, Comfere N I. Programmed death 1 is expressed in cutaneous infiltrates of mycosis fungoides and Sézary syndrome. Am J Hematol. 2011; 86(3):325-327. doi:10.1002/ajh.21960
9. Klemke C D, Booken N, Weiss C, et al. Histopathological and immunophenotypical criteria for the diagnosis of Sézary syndrome in differentiation from other erythrodermic skin diseases: a European Organisation for Research and Treatment of Cancer (EORTC) Cutaneous Lymphoma Task Force Study of 97 cases. Br J Dermatol. 2015; 173(1):93-105. doi:10.1111/bjd.13832
10. Moins-Teisserenc H, Daubord M, Clave E, et al. CD158k is a reliable marker for diagnosis of Sézary syndrome and reveals an unprecedented heterogeneity of circulating malignant cells. J Invest Dermatol. 2015; 135(1):247-257. doi:10.1038/jid.2014.356
11. Prince H M, Kim Y H, Horwitz S M, et al. Brentuximab vedotin or physician's choice in CD30-positive cutaneous T-cell lymphoma (ALCANZA): an international, open-label, randomised, phase 3, multicentre trial. Lancet Lond Engl. 2017; 390(10094):555-566. doi:10.1016/S0140-6736(17)31266-7
12. Kim Y H, Bagot M, Pinter-Brown L, et al. Mogamulizumab versus vorinostat in previously treated cutaneous T-cell lymphoma (MAVORIC): an international, open-label, randomised, controlled phase 3 trial. Lancet Oncol. 2018; 19(9):1192-1204. doi:10.1016/S1470-2045(18)30379-6
13. Bagot M, Porcu P, Marie-Cardine A, et al. IPH4102, a first-in-class anti-KIR3DL2 monoclonal antibody, in patients with relapsed or refractory cutaneous T-cell lymphoma: an international, first-in-human, open-label, phase 1 trial. Lancet Oncol. 2019; 20(8):1160-1170. doi:10.1016/S1470-2045(19)30320-1
14. Upadhyay R, Hammerich L, Peng P, Brown B, Merad M, Brody J D. Lymphoma: immune evasion strategies. Cancers. 2015; 7(2):736-762. doi:10.3390/cancers7020736
15. Schietinger A, Greenberg P D. Tolerance and exhaustion: defining mechanisms of T cell dysfunction. Trends Immunol. 2014; 35(2):51-60. doi:10.1016/j.it.2013.10.001
16. Querfeld C, Leung S, Myskowski P L, et al. Primary T Cells from Cutaneous T-cell Lymphoma Skin Explants Display an Exhausted Immune Checkpoint Profile. Cancer Immunol Res. 2018; 6(8):900-909. doi:10.1158/2326-6066.CIR-17-0270
17. Bosisio F M, Cerroni L. Expression of T-follicular helper markers in sequential biopsies of progressive mycosis fungoides and other primary cutaneous T-cell lymphomas. Am J Dermatopathol. 2015; 37(2):115-121. doi:10.1097/DAD.0000000000000258
18. Amatore F, Gorvel L, Olive D. Inducible Co-Stimulator (ICOS) as a potential therapeutic target for anti-cancer therapy. Expert Opin Ther Targets. 2018; 22(4):343-351. doi:10.1080/14728222.2018.1444753
19. Amatore F, Gorvel L, Olive D. Role of Inducible Co-Stimulator (ICOS) in cancer immunotherapy. Expert Opin Biol Ther. November 2019:1-10. doi:10.1080/14712598.2020.1693540
20. Marafioti T, Paterson J C, Ballabio E, et al. The inducible T-cell co-stimulator molecule is expressed on subsets of T cells and is a new marker of lymphomas of T follicular helper cell-derivation. Haematologica. 2010; 95(3):432-439. doi:10.3324/haematol.2009.010991
21. Buonfiglio D, Bragardo M, Bonissoni S, et al. Characterization of a novel human surface molecule selectively expressed by mature thymocytes, activated T cells and subsets of T cell lymphomas. Eur J Immunol. 1999; 29(9):2863-2874. doi:10.1002/(SICI)1521-4141(199909)29:09& #60; 2863::AID-IMMIVU2863& #62; 3.0.CO; 2-W
22. Downs-Canner S, Berkey S, Delgoffe G M, et al. Suppressive IL-17A+Foxp3+ and ex-Th17 IL-17AnegFoxp3+ Treg cells are a source of tumour-associated Treg cells. Nat Commun. 2017; 8:14649. doi:10.1038/ncomms14649
23. Geskin L J, Akilov O E, Kwon S, et al. Therapeutic reduction of cell-mediated immunosuppression in mycosis fungoides and Sézary syndrome. Cancer Immunol Immunother CII. 2018; 67(3):423-434. doi:10.1007/s00262-017-2090-z
24. Ni X, Jorgensen J L, Goswami M, et al. Reduction of regulatory T cells by Mogamulizumab, a defucosylated anti-CC chemokine receptor 4 antibody, in patients with aggressive/refractory mycosis fungoides and Sézary syndrome. Clin Cancer Res Off J Am Assoc Cancer Res. 2015; 21(2):274-285. doi:10.1 158/1078-0432.CCR-14-0830
25. Willemze R, Cerroni L, Kempf W, et al. The 2018 update of the WHO-EORTC classification for primary cutaneous lymphomas. Blood. 2019; 133(16):1703-1714. doi:10.1 182/blood-2018-11-881268
26. Olsen E A, Whittaker S, Kim Y H, et al. Clinical end points and response criteria in mycosis fungoides and Sézary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol Off J Am Soc Clin Oncol. 2011; 29(18):2598-2607. doi:10.1200/JCO.2010.32.0630
27. Le K-S, Thibult M-L, Just-Landi S, et al. Follicular B Lymphomas Generate Regulatory T Cells via the ICOS/ICOSL Pathway and Are Susceptible to Treatment by Anti-ICOS/ICOSL Therapy. Cancer Res. 2016; 76(16):4648-4660. doi:10.1158/0008-5472.CAN-15-0589
28. Townsend E C, Murakami M A, Christodoulou A, et al. The Public Repository of Xenografts Enables Discovery and Randomized Phase II-like Trials in Mice. Cancer Cell. 2016; 29(4):574-586. doi:10.1016/j.ccell.2016.03.008
29. Rubio Gonzalez B, Zain J, Rosen S T, Querfeld C. Tumor microenvironment in mycosis fungoides and Sézary syndrome. Curr Opin Oncol. 2016; 28(1):88-96. doi:10.1097/CCO.0000000000000243
30. Nelson M H, Kundimi S, Bowers J S, et al. The inducible costimulator augments Tc17 cell responses to self and tumor tissue. J Immunol Baltim Md 1950. 2015; 194(4):1737-1747. doi:10.4049/jimmunol.1401082
31. Faget J, Bendriss-Vermare N, Gobert M, et al. ICOS-ligand expression on plasmacytoid dendritic cells supports breast cancer progression by promoting the accumulation of immunosuppressive CD4+ T cells. Cancer Res. 2012; 72(23):6130-6141. doi:10.1158/0008-5472.CAN-12-2409
32. Nagase H, Takeoka T, Urakawa S, et al. ICOS+Foxp3+ TILs in gastric cancer are prognostic markers and effector regulatory T cells associated with Helicobacter pylori. Int J Cancer. 2017; 140(3):686-695. doi:10.1002/ijc.30475
33. Giraldo N A, Becht E, Vano Y, et al. Tumor-Infiltrating and Peripheral Blood T-cell Immunophenotypes Predict Early Relapse in Localized Clear Cell Renal Cell Carcinoma. Clin Cancer Res Off J Am Assoc Cancer Res. 2017; 23(15):4416-4428. doi:10.1158/1078-0432.CCR-16-2848
34. Iliadis A, Koletsa T, Patsatsi A, Georgiou E, Sotiriadis D, Kostopoulos I. The cellular microenvironment and neoplastic population in mycosis fungoides skin lesions: a clinicopathological correlation. Eur J Dermatol EJD. 2016; 26(6):566-571. doi:10.1684/ejd.2016.2847
35. Marie-Cardine A, Viaud N, Thonnart N, et al. IPH4102, a humanized KIR3DL2 antibody with potent activity against cutaneous T-cell lymphoma. Cancer Res. 2014; 74(21):6060-6070. doi:10.1158/0008-5472.CAN-14-1456
36. Wallin J J, Liang L, Bakardjiev A, Sha W C. Enhancement of CD8+ T cell responses by ICOS/B7h costimulation. J Immunol Baltim Md 1950. 2001; 167(1):132-139.
37. Klingenberg R, Autschbach F, Gleissner C, et al. Endothelial inducible costimulator ligand expression is increased during human cardiac allograft rejection and regulates endothelial cell-dependent allo-activation of CD8+ T cells in vitro. Eur J Immunol. 2005; 35(6):1712-1721. doi:10.1002/eji.200425727
38. Schenk A D, Gorbacheva V, Rabant M, Fairchild R L, Valujskikh A. Effector functions of donor-reactive CD8 memory T cells are dependent on ICOS induced during division in cardiac grafts. Am J Transplant Off J Am Soc Transplant Am Soc Transpl Surg. 2009; 9(1):64-73. doi:10.1111/j.1600-6143.2008.02460.x
39. Dan N, Setua S, Kashyap V K, et al. Antibody-Drug Conjugates for Cancer Therapy: Chemistry to Clinical Implications. Pharm Basel Switz. 2018; 11(2). doi:10.3390/ph11020032
40. Khera E, Thurber G M. Pharmacokinetic and Immunological Considerations for Expanding the Therapeutic Window of Next-Generation Antibody-Drug Conjugates. BioDrugs Clin Immunother Biopharm Gene Ther. August 2018. doi:10.1007/s40259-018-0302-5
41. Lemonnier F, Couronne L, Parrens M, et al. Recurrent TET2 mutations in peripheral T-cell lymphomas correlate with T_{FH}-like features and adverse clinical parameters. Blood. 2012; 120(7):1466-1469. doi:10.1182/blood-2012-02-408542
42. Mourad N, Mounier N, Briere J, et al. Clinical, biologic, and pathologic features in 157 patients with angioimmunoblastic T-cell lymphoma treated within the Groupe d'Etude des Lymphomes de l'Adulte (GELA) trials. Blood. 2008; 111(9):4463-4470. doi:10.1 182/blood-2007-08-105759.
Solinas Cinzia, Chunyan Gu-Trantien, Karen Willard-Gallo. The rationale behind targeting the ICOS-ICOS ligand costimulatory pathway in cancer immunotherapy.
Scarfó I, Frigault M J, Maus M V. CAR-Based Approaches to Cutaneous T-Cell Lymphoma. Front Oncol. 2019 Apr. 16; 9:259. doi: 10.3389/fonc.2019.00259. eCollection 2019.
Shimin Hu, Ken H. Young, Sergej N. Konoplev and Jeffrey Medeiros. Follicular T-cell lymphoma: a member of an emerging family of follicular helper T-cell derived T-cell lymphomas. Human Pathology, Volume 43, Issue 11, November 2012, Pages 1789-1798.

1. A method of treating a cutaneous T-cell lymphomas (CTCL) and/or a T_{FH} derived lymphoma in a subject in need thereof comprising, administering to the subject a therapeutically effective amount of an anti ICOS antibody.

2. The method according to the claim 1, wherein the T_{FH} derived lymphoma is an angioimmunoblastic T-cell Lymphoma (AITL).

3. The method according to claim 1, wherein the CTCL is a mycosis fungoides or a Sézary syndrome.

4. The method according to claim 1, wherein the antibody is the 53.3 mab, the 88.2 mab, the 92.17 mab, the 145.1 mab or the 314.8 mab.

5. The method according to claim 1, wherein the antibody is used in an antibody-drug conjugate (ADC) or antibody-dependent cell-mediated cytotoxicity (ADCC) and/or antibody-dependent cellular phagocytosis (ADCP).

6. The method according to claim 1, wherein said antibody is conjugated to a cytotoxic moiety.

7. The method according to claim 6 wherein said cytotoxic moiety is selected from the group consisting of taxol;
cytochalasin B; gramicidin D; ethidium bromide; emetine; mitomycin; etoposide;
tenoposide; vincristine; vinblastine; colchicin; doxorubicin; daunorubicin; dihydroxy anthracin dione; a tubulin-inhibitor; an antimitotic agent; dolastatin 10 or 15; irinotecan; mitoxantrone; mithramycin; actinomycin D; 1-dehydrotestosterone; a glucocorticoid; procaine; tetracaine; lidocaine; propranolol; puromycin; calicheamicin; an antimetabolite; an alkylating agent; a platinum derivative; duocarmycin A, duocarmycin SA, rachelmycin (CC-1065; an antibiotic; pyrrolo[2,1-c][1,4]-benzodiazepines (PDB); diphtheria toxin, ricin toxin, cholera toxin, a Shiga-like toxin, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; and an amatoxin.

8. The method according to claim 7, wherein said cytotoxic moiety is MMAE.

9. (canceled)

10. (canceled)

11. The method of claim 7, wherein
the tubulin-inhibitor is maytansine;
the antimitotic agent is monomethyl auristatin E or F (MMAE or MMAF);
the antimetabolite is methotrexate, 6 mercaptopurine, 6 thioguanine, cytarabine, fludarabin, 5 fluorouracil, decarbazine, hydroxyurea, asparaginase, gemcitabine, or cladribine;
the alkylating agent is mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine or mitomycin C;
the platinum derivative is cisplatin or carboplatin;
the antibiotic is dactinomycin, bleomycin, daunorubicin, doxorubicin, idarubicin, mithramycin, mitomycin, mitoxantrone, plicamycin or anthramycin (AMC);
the ricin toxin is ricin A or a deglycosylated ricin A chain toxin;
the Shiga-like toxin is SLT I, SLT II, SLT of IIV;
the Phytolacca americana protein is PAPI, PAPII or PAP-S; and
the amatoxin is α-amanitin, β-amanitin, γ-amanitin, ε-amanitin, amanullin, amanullinic acid, amaninamide, amanin or aroamanullin.


SEQUENCE LISTING (32 sequences)

SEQ ID NO: 1 - protein, 8 aa, Artificial
  Synthetic 88.2 antibody - H-CDR1 - Amino acids sequence (IMGT)
GYSFTSYW

SEQ ID NO: 2 - protein, 8 aa, Artificial
  Synthetic 88.2 antibody - H-CDR2 - Amino acids sequence (IMGT)
IYPSDSYT

SEQ ID NO: 3 - protein, 17 aa, Artificial
  Synthetic 88.2 antibody - H-CDR3 - Amino acids sequence (IMGT)
TRWNLSYYFDNNYYLDY

SEQ ID NO: 4 - protein, 11 aa, Artificial
  Synthetic 88.2 antibody - L-CDR1 - Amino acids sequence (IMGT)
KSLLHSNGNTY

SEQ ID NO: 5 - protein, 3 aa, Artificial
  Synthetic 88.2 antibody - L-CDR2 - Amino acids sequence (IMGT)
RMS

SEQ ID NO: 6 - protein, 9 aa, Artificial
  Synthetic 88.2 antibody - L-CDR3 - Amino acids sequence (IMGT)
MQHLEYPWT

SEQ ID NO: 7 - protein, 124 aa, Artificial
  Synthetic Amino acids sequence of the heavy chain (H) of the 88.2 mAb
QVQLQQPGAELVRPGASVKLSCKASGYSFTSYWINWVKQRPGQGLEWIGNIYPSDSYTNYNQMFKDKATLTVDKSSNTAYMQLTSPTSEDSAVYYCTRWNLSYYFDNNYYLDYWGQGTTLTVSS

SEQ ID NO: 8 - protein, 112 aa, Artificial
  Synthetic Amino acids sequence of the light chain (L) of the 88.2 mAb
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPWTFGGGTKLEIK

SEQ ID NO: 9 - protein, 8 aa, Artificial
  Synthetic 314.8 antibody - H-CDR1 - Amino acids sequence (IMGT)
GYTFTTYW

SEQ ID NO: 10 - protein, 8 aa, Artificial
  Synthetic 314.8 antibody - H-CDR2 - Amino acids sequence (IMGT)
IDPSDSYV

SEQ ID NO: 11 - protein, 16 aa, Artificial
  Synthetic 314.8 antibody - H-CDR3 - Amino acids sequence (IMGT)
ARSPDYYGTSLAWFDY

SEQ ID NO: 12 - protein, 11 aa, Artificial
  Synthetic 314.8 antibody - L-CDR1 - Amino acids sequence (IMGT)
KSPLHSNGNIY

SEQ ID NO: 13 - protein, 3 aa, Artificial
  Synthetic 314.8 antibody - L-CDR2 - Amino acids sequence (IMGT)
RMS

SEQ ID NO: 14 - protein, 9 aa, Artificial
  Synthetic 314.8 antibody - L-CDR3 - Amino acids sequence (IMGT)
MQHLEYPYT

SEQ ID NO: 15 - protein, 123 aa, Artificial
  Synthetic Amino acids sequence of the heavy chain (H) of the 314.8 mAb
QVQLQQPGTELMKPGASVKLSCKASGYTFTTYWMHWVKQRPGQGLEWIGEIDPSDSYVNYNQNFKGKATLTVDKSSSTAYIQLSSLTSEDSAVYFCARSPDYYGTSLAWFDYWGQGTLVTVST

SEQ ID NO: 16 - protein, 112 aa, Artificial
  Synthetic Amino acids sequence of the light chain (L) of the 314.8 mAb
DIVMTQAAPSVPVTPGESVSISCRSSKSPLHSNGNIYLYWFLQRPGQSPQLLIYRMSNLASGVPDRFSGSGSGTTFTLKISRVEAEDVGVYYCMQHLEYPYTFGGGTKLEIK

SEQ ID NO: 17 - DNA, 372 bp, Artificial
  Synthetic Nucleic acids sequence of the heavy chain (H) of the 88.2 mAb
caggtccaactgcagcagcctggggctgagctggtgaggcctggggcttcagtgaagctgtcctgcaaggcttctggctacagtttcaccagctactggataaactgggtgaagcagaggcctggacaaggccttgagtggatcggaaatatttatccttctgatagttatactaactacaatcaaatgttcaaggacaaggccacattgactgtagacaaatcctccaacacagcctacatgcagctcaccagcccgacatctgaggactctgcggtctattactgtacaagatggaatctttcttattacttcgataataactactacttggactactggggccaaggcaccactctcacagtctcctca

SEQ ID NO: 18 - DNA, 336 bp, Artificial
  Synthetic Nucleic acids sequence of the light chain (L) of the 88.2 mAb
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcaactcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtggacgttcggtggaggcaccaagctggaaatcaaa

SEQ ID NO: 19 - DNA, 369 bp, Artificial
  Synthetic Nucleic acids sequence of the heavy chain (H) of the 314.8 mAb
caggtccaactacagcagcctgggactgaacttatgaagcctggggcttcagtgaagctgtcctgcaaggcttctggctacaccttcaccacctactggatgcactgggtgaagcagaggcctggacaaggccttgagtggatcggagagattgatccttctgatagttatgttaactacaatcaaaactttaagggcaaggccacattgactgtagacaaatcctccagcacagcctacatacagctcagcagcctgacatctgaggactctgcggtctatttttgtgcgagatcccctgattactacggtactagtcttgcctggtttgattactggggccaagggactctggtcactgtctctaca

SEQ ID NO: 20 - DNA, 336 bp, Artificial
  Synthetic Nucleic acids sequence of the light chain (L) of the 314.8 mAb
gatattgtgatgactcaggctgcaccctctgtacctgtcactcctggagagtcagtatccatctcctgcaggtctagtaagagtcccctgcatagtaacggcaacatttacttatattggttcctgcagaggccaggccagtctcctcagctcctgatatatcggatgtccaaccttgcctcaggagtcccagacaggttcagtggcagtgggtcaggaactactttcacactgaaaatcagtagagtggaggctgaggatgtgggtgtttattactgtatgcaacatctagaatatccgtacacgttcggaggggggaccaagctggaaataaaa

SEQ ID NO: 21 - protein, 5 aa, Artificial
  Synthetic 88.2 antibody - H-CDR1 - Amino acids sequence (KABAT)
SYWIN

SEQ ID NO: 22 - protein, 17 aa, Artificial
  Synthetic 88.2 antibody - H-CDR2 - Amino acids sequence (KABAT)
NIYPSDSYTNYNQMFKD

SEQ ID NO: 23 - protein, 15 aa, Artificial
  Synthetic 88.2 antibody - H-CDR3 - Amino acids sequence (KABAT)
WNLSYYFDNNYYLDY

SEQ ID NO: 24 - protein, 16 aa, Artificial
  Synthetic 88.2 antibody - L-CDR1 - Amino acids sequence (KABAT)
RSSKSLLHSNGNTYLY

SEQ ID NO: 25 - protein, 7 aa, Artificial
  Synthetic 88.2 antibody - L-CDR2 - Amino acids sequence (KABAT)
RMSNLAS

SEQ ID NO: 26 - protein, 9 aa, Artificial
  Synthetic 88.2 antibody - L-CDR3 - Amino acids sequence (KABAT)
MQHLEYPWT

SEQ ID NO: 27 - protein, 5 aa, Artificial
  Synthetic 314.8 antibody - H-CDR1 - Amino acids sequence (KABAT)
TYWMH

SEQ ID NO: 28 - protein, 17 aa, Artificial
  Synthetic 314.8 antibody - H-CDR2 - Amino acids sequence (KABAT)
EIDPSDSYVNYNQNFKG

SEQ ID NO: 29 - protein, 14 aa, Artificial
  Synthetic 314.8 antibody - H-CDR3 - Amino acids sequence (KABAT)
SPDYYGTSLAWFDY

SEQ ID NO: 30 - protein, 16 aa, Artificial
  Synthetic 314.8 antibody - L-CDR1 - Amino acids sequence (KABAT)
RSSKSPLHSNGNIYLY

SEQ ID NO: 31 - protein, 7 aa, Artificial
  Synthetic 314.8 antibody - L-CDR2 - Amino acids sequence (KABAT)
RMSNLAS

SEQ ID NO: 32 - protein, 9 aa, Artificial
  Synthetic 314.8 antibody - L-CDR3 - Amino acids sequence (KABAT)
MQHLEYPYT